(19) United States
(12) Patent Application Publication
(10) Pub. No.: US 2025/0195446 A1
(43) Pub. Date: Jun. 19, 2025

(54) ANTIBACTERIAL DRUG-ELUTING COMPOSITIONS AND METHODS

(71) Applicant: THE REGENTS OF THE UNIVERSITY OF CALIFORNIA, Oakland, CA (US)

(72) Inventors: Nasim Annabi, Los Angeles, CA (US); Shima Gholizadeh, Los Angeles, CA (US); Yangcheng Liu, Shenzhen (CN)

(21) Appl. No.: 18/846,546

(22) PCT Filed: Mar. 16, 2023
(86) PCT No.: PCT/US2023/064544

§ 371 Date: Sep. 12, 2024

Related U.S. Application Data
(60) Provisional application No. 63/320,328, filed on Mar. 16, 2022.

(51) Int. Cl. A61K 9/51; A61K 9/00; A61K 9/06; A61P 31/04

(52) U.S. Cl. CPC A61K 9/5169; A61K 9/0048; A61K 9/5192; A61P 31/04; A61K 9/06

(57) ABSTRACT

The present disclosure describes compositions that can be used in the treatment of an ocular injury or disease in a subject in need thereof. Methods of preparing the compositions are also described. The compositions can include one or more nanoparticles encapsulating a hydrophilic therapeutic agent; gelatin methacryloyl (GelMA); hyaluronic acid-glycidyl methacrylate (HAGM); and a visible light-activated photoinitiator.

### FEDERALLY SPONSORED RESEARCH

This invention was made with government support under W81XWH-18-1-0654, awarded by the U.S. Department of Defense. The government has certain rights in the invention.

### TECHNICAL FIELD

The present disclosure describes compositions comprising photocrosslinkable gelatin-based composite adhesive hydrogels incorporating gelatin-based nanoparticles. The disclosure also describes methods of treating an ocular injury or ocular disease in a subject in need thereof with these compositions and methods of preparing the compositions. The compositions can include gelatin-based nanoparticles (NPs), gelatin methacryloyl (GelMA), hyaluronic acid-glycidyl methacrylate (HAGM), and a visible light-activated photoinitiator. The methods can include contacting the eye of the subject with the composition and photo-crosslinking the composition by exposing the composition to a visible light.

### BACKGROUND

There is an increase in the prevalence of eye diseases such as glaucoma, dry eye disease and age-related macular degeneration. This may be correlated with the increase of an aging population, changes in lifestyle, and prolonged wear of contact lenses worldwide. Apart from chronic disease-based eye injuries, acute physical injuries, spanning from superficial abrasions to full-thickness perforations, can result from a variety of causes including blunt force injuries, penetration of foreign bodies, and chemical burns, among others.

In general, ocular-induced primary injuries due to either chronic disease or physical abrasions are associated with an elevated risk of infection due to breakdown and/or rupture of the main barrier (e.g., surface epithelium layer) on the cornea or sclera, which may permit infiltration of microorganism into the eye. Bacterial infection is one of the main causes of many eye-related inflammations which can lead to conjunctivitis, keratitis, and endophthalmitis, thus routinely requires an antibiotic-based therapeutic intervention.

Bacterial keratitis is caused by bacterial (e.g., Pseudomonas aeruginosa and Staphylococcus aureus) infection in the cornea. Conjunctivitis, a common eye disease, is caused by inflammation of a thin semi-transparent membrane on the inner surface of eyelids due to the bacterial infection. Nearly 6 million people are affected by acute conjunctivitis in the United States each year, and an average of $617 million is spent on the treatment of bacterial conjunctivitis annually by U.S. medical institutions.

Topical ophthalmic antibiotics are the most commonly used drug administration route via topical instillation. Topical ophthalmic antibiotics are often applied to relieve the inflammation symptoms and facilitate recovering process. However, in some cases, ophthalmic antibiotics must be administered via direct injection to the eye through a specialized needle. The topical instillation has poor bioavailability due to mechanisms such as reflex blinking and tear-film turnover. Due to the presence of static and dynamic ocular barriers, less than about 5% of a dose can be delivered to the posterior segment of eyes using conventional administration routes.

To overcome these challenges, various ocular drug delivery methods such as periocular and intravitreal injection and oral and subconjunctival administration can be used. Unlike topical instillation, intravitreal injection can be used to apply drugs directly in the affected area in the eye. However, these methods may not be patient compliant and can cause serious side effects including hemorrhage and retinal detachments. An oral administration route, on the other hand, is noninvasive, but, a high dosage is required to achieve a therapeutic effect, which can further lead to toxicity and undesired side effects. Thus, there is a need for a localized, noninvasive, and efficient ocular drug delivery vehicle and method capable of having a high patient compliance.

### SUMMARY

Certain aspects of the present disclosure are directed to a composition comprising: one or more nanoparticles comprising gelatin methacryloyl (GelMA) and encapsulating a hydrophilic therapeutic agent; GelMA; hyaluronic acid-glycidyl methacrylate (HAGM); and a visible light-activated photoinitiator.

In some embodiments, the one or more nanoparticles comprises GelMA at a concentration of about 5% (w/v) to about 30% (w/v). In some embodiments, the GelMA present in the composition comprises a degree of methylation of about 40% to about 85%. In some embodiments, the one or more nanoparticles is chemically crosslinked and photo-crosslinked. In some embodiments, the one or more nanoparticles is chemically crosslinked with glutaraldehyde at concentration of about 0.04% (w/v). In some embodiments, the hydrophilic therapeutic agent is a first therapeutic agent, and the composition further comprises a second therapeutic agent. In some embodiments, the hydrophilic therapeutic agent is present at a concentration of about 0.01% (w/v) to about 1% (w/v). In some embodiments, the hydrophilic therapeutic agent is present at a concentration of about 0.25 μg/mL to about 5 mg/ml. In some embodiments, the one or more nanoparticles are encapsulating the hydrophilic therapeutic agent at an encapsulation efficiency of at least about 85%. In some embodiments, the hydrophilic therapeutic agent is an antibacterial agent. In some embodiments, the antibacterial agent comprises antibiotic drugs, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, chitosan, or any combination thereof. In some embodiments, the one or more nanoparticles have a diameter ranging from about 150 nanometers (nm) to about 250 nm. In some embodiments, the one or more nanoparticles have a polydispersity index (PDI) of less than about 0.2. In some embodiments, the composition has a compressive modulus of about of about 15 kPa to about 50 kPa. In some embodiments, the composition has a tensile modulus of about 15 kPa to about 50 kPa. In some embodiments, the composition has a burst pressure of about 10 kPa to about 50 kPa. In some embodiments, the GelMA is present in the composition at a concentration of about 3% (w/v) to about 14% (w/v). In some embodiments, the HAGM is present in the composition at a concentration of about 0.5% (w/v) to about 3% (w/v). In some embodiments, the composition has a sustained drug release profile over a period of at least about 5 days. In some embodiments, the photoinitiator comprises Eosin Y, triethanolamine (TEA), N-vinylcaprolactam (VC), or any combination thereof. In some embodiments, the composition is in a form of a solution or a hydrogel. In some embodiments, the composition further comprises a pharmaceutically acceptable carrier or excipient. In some embodiments, the composition is formulated for topical use.

Certain aspects of the present disclosure are directed to a method of treating an ocular disease or an ocular injury in an eye of a subject, the method comprising: contacting the eye of the subject with any of the compositions of the disclosure and photocrosslinking the composition by exposing the composition to a visible light.

Certain aspects of the present disclosure are directed to a method of preparing the composition of any one of claims 1-23, the method comprising: synthesizing the one or more nanoparticles; dissolving the GelMA and the HAGM in a solution comprising the visible light-activated photoinitiator; and mixing the one or more nanoparticles with the dissolved GelMA and HAGM in the solution.

In some embodiments, the solution is a first solution, and wherein synthesizing the one or more nanoparticles comprises: mixing a chemical crosslinker and a photoinitiator with a second solution comprising GelMA while constantly stirring; and exposing the one or more nanoparticles to an ultraviolet light, thereby photocrosslinking the one or more nanoparticles.

In some embodiments, the chemical crosslinker is present at a concentration of about 0.1% (w/v) to about 1% (w/v). In some embodiments, the chemical crosslinker is glutaraldehyde. In some embodiments, the method further comprises mixing a therapeutic agent with the second solution prior to mixing the second solution with the chemical crosslinker and the photoinitiator. In some embodiments, the first and second solutions further comprise a buffer, a solvent, water, or any combination thereof. In some embodiments, the method further comprises photocrosslinking the solution after mixing the one or more nanoparticles with the dissolved GelMA and HAGM by exposing the composition to a visible light. In some embodiments, the composition changes from a solution form to a hydrogel form after photocrosslinking.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

As used herein, the term “nanoparticle” refers to particles (e.g., gelatin-based particles) having at least one dimension (e.g., a diameter) ranging from about 100 nm to about 400 nm (e.g., between about 130 nm to about 350, between 100 nm and 200 nm, or between 150 nm and 200 nm). Non-limiting examples of nanoparticles include the nanoparticles described herein.

As used in the specification and the appended claims, the singular forms “a,” “an,” and “the” include plural referents unless the context clearly dictates otherwise. Thus, for example, reference to “a nanoparticle” includes mixtures of nanoparticles, reference to “a nanoparticle” includes mixtures of two or more such nanoparticles, and the like.

The term “ocular surface injury,” as used herein, can include ulcers, lacerations, defects, perforations, or intentionally performed incisions (e.g., as is done in surgery) of the cornea or sclera.

As used herein, the term “therapeutic agent” is any molecule or atom that is encapsulated, conjugated, fused, dispersed, embedded, mixed, or otherwise affixed to any of the compositions described herein and is useful for a disease therapy.

As used herein, the expression “pharmaceutically acceptable” applies to a composition which contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. A composition as described herein can comprise one or more carriers, useful excipients, and/or diluents.

As used herein, the term “hydrogel” refers to a broad class of polymeric materials that may be natural or synthetic, have an affinity for an aqueous medium, and are able to absorb large amounts of the aqueous medium, but which do not normally dissolve in the aqueous medium.

As used herein, the term “aqueous medium” as used herein refers to water or a solution based primarily on water such as phosphate-buffered saline (PBS), or water containing one or more salts dissolved therein.

As used herein, the term “photo-crosslink” refers to an interconnection between polymer chains via chemical bonding, such as, but not limited to, covalent bonding, ionic bonding, or affinity interactions that are caused by exposure to a light source. The chemical cross-linking can be carried out by reactions, such as any one of free radical polymerization, condensation polymerization, anionic or cationic polymerization, or step growth polymerization.

As used herein, the term “biodegradable” refers to a substance which may be broken down by microorganisms, or which spontaneously breaks down over a relatively short time (within about 14 days to about 6 months) when exposed to environmental conditions commonly found in nature. For example, the compositions described herein may be degraded by enzymes that are present in the body (e.g., the ocular environment).

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Furthermore, the use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A-1B show the synthesis and application of GelPatch adhesive hydrogels loaded with moxifloxacin (MXF)-loaded GelMA NPs. FIG. 1A is a stepwise schematic of the preparation procedure of MXF-loaded GelMA NPs. FIG. 1B is a schematic of the formation and application for MXF-loaded GelMA NPs incorporated GelPatch.

FIGS. 2A-2G show the physicochemical characterization of gelatin/GelMA NPs. Effect of polymer type and concentration on hydrodynamic size (FIG. 2A), PDI (FIG. 2B), and zeta potential of the NPs (FIG. 2C) are shown in FIGS. 2A-2C, respectively. Effect of GA concentration on hydrodynamic size (FIG. 2D) and PDI (FIG. 2E) of the NPs are shown in FIGS. 2D-2E, respectively. FIG. 2F is a group of photographic images showing a visual comparison among NP solutions made of different polymer and concentration. FIG. 2G is a group of photographic images showing a Visual comparison among NP solutions made of GelMA and various GA concentrations. Data are reported as means±SD (*: P<0.05, **: P<0.01, ***: P<0.001, ****: P<0.0001; n>3).

FIGS. 3A-3F show the physicochemical characterization of MXF-loaded GelMA NPs (the particle was formed using 10% H-GelMA/0.4% GA with different amount of MXF). Effect of drug loading on hydrodynamic size (FIG. 3A), PDI (FIG. 3B), and surface charge density (FIG. 3C) of the NPs are shown in FIGS. 3A-3C, respectively. Effect of drug loading on loaded MXF concentration of NPs (FIG. 3D) and MXF loading efficiency of NPs (FIG. 3E) are shown in FIGS. 3D-3E, respectively. FIG. 3F is a representative TEM image of MXF-loaded NPs in DI water. Data are reported as means±SD (*: P<0.05, **: P<0.01, ***: P<0.001, ****: P<0.0001; n>3).

FIGS. 4A-4C show in vitro cumulative release profile and kinetics. FIG. 4A is a graph showing an in vitro cumulative release study of 0.05%, 0.25%, 0.5% and 1% (w/v) of MXF-loaded GelMA NPs. FIG. 4B is a graph showing an in vitro cumulative release study of MXF-loaded NPs incorporated GelPatch and free MXF-loaded GelPatch. FIG. 4C is a graph showing the release kinetics for free MXF-loaded GelPatch and MXF-loaded GelMA NPs incorporated GelPatch (fraction of MXF released vs. square root of time over 5 days.

FIGS. 5A-5I show the physical characterization for three GelPatch formulations (GelPatch, GelPatch loaded with free MXF and MXF-loaded GelMA NPs incorporated GelPatch). FIGS. 5A and 5B are graphs showing the compressive modulus and compressive ultimate stress, respectively, of the hydrogels. FIG. 5C is a group of two photographic images of free MXF-loaded GelPatch and MXF-loaded GelMA NPs incorporated GelPatch. FIGS. 5D-5F are graphs showing the tensile modulus, tensile ultimate stress, and tensile ultimate strain, respectively, of the hydrogels. FIG. 5G is a graph showing the burst pressure, and FIG. 5H is a graph showing the swelling ratio after a 24 hr-immersion in DPBS solution. FIG. 5I is a photographic image showing an exemplary burst pressure instrument setup.

FIGS. 6A-6D show in vitro biocompatibility and cytotoxicity tests. FIG. 6A is a group of fluorescence microscopy images, and FIG. 6B is a graph showing the cell viability of a Live/Dead™ assay and a PrestoBlue™ assay, at days 1 and 3 for GelPatch and MXF-loaded GelMA NPs incorporated GelPatch. FIG. 6C is a group of fluorescence microscopy images of fluorescent staining F-actin for cytoskeleton of cells after 1 and 3 days of post-seeding. FIG. 6D is a graph showing the fluorescence level (representing the enzymatic activity) of cells after 1, 3, and 7 days of post-seeding.

FIGS. 7A-7D shows the in vitro antibacterial effects of MXF-loaded GelMA NPs incorporated GelPatch. FIG. 7A is a group of selected photographic images of bacteria grown on agar plates for GelPatch with and without MXF-loaded GelMA NPs. FIGS. 7B and 7C are graphs showing the quantitative characterization for ZOI with GelMA NPs incorporated GelPatch placed on agar plates seeded with Pseudomonas aeruginosa and Staphylococcus aureus bacteria, respectively. FIG. 7D is a graph showing the quantitative characterization of CFU concentration for GelPatch with MXF-loaded GelMA NPs placed on agar plates seeded with Pseudomonas aeruginosa and Staphylococcus aureus bacteria.

### DETAILED DESCRIPTION

The compositions described herein include biocompatible, photocrosslinkable gelatin-based adhesive hydrogels including one or more polymeric nanoparticles. In some examples, the compositions described herein are used for targeted ocular drug delivery. Methods of using and/or preparing these compositions are also provided herein. Some embodiments of the compositions and methods described herein may provide one or more of the following advantages.

Certain embodiments of the present disclosure include biocompatible, photocrosslinkable gelatin-based adhesive hydrogels. As discussed above, there is currently an unmet need for a localized, noninvasive, and efficient ocular drug delivery vehicle that could have a high patient compliance. The compositions and methods of the present disclosure address this need. For example, in some embodiments, the compositions and methods described herein can achieve a sustained drug release profile for an extended period of time (e.g., about 5 to about 15 days). Furthermore, the compositions of the disclosure may facilitate drug penetration through the structural barriers of ocular tissues by adhering to the ocular surface and providing a sustained release of drugs directly to the injured sites. Thus, in some embodiments, a unique property of the compositions and methods described herein is its ability to provide localized and sustained delivery of one or more therapeutic agents directly in and/or onto the eye. Furthermore, in some embodiments, the compositions and methods described herein enable the delivery of hydrophilic therapeutic agents (e.g., antibiotics) that can be encapsulated in the nanoparticles.

Some embodiments described herein may provide a non-invasive, adhesive, and biocompatible drug-eluting hydrogel patch that can provide lower dosage requirements to ensure better patient compliance. Thus, in some embodiments, the compositions and methods of the disclosure may offer a better alternative to current treatments, which are often invasive (e.g., injecting a therapeutic into the eye) or have low patient compliance (e.g., require a patient to self-administer one or more eye drops multiple times a day for an extended period of time).

Some embodiments described herein may provide site-targeted delivery to the eye. For example, systemic administration routes may require a large dose in order to achieve a satisfactory drug concentration at the ocular tissue, which can lead to off-target systemic side effects. On the other hand, local drug delivery such as the conventional topical administration (eye drops or ointments) have extremely low bioavailability (e.g., of about less than 5%) due to corneal epithelium barrier and fast clearance by tear film and blinking. As a result, repetitive drug applications may be required, which may induce ocular hypertension and are also associated with poor patient compliance. Furthermore, in some embodiments, the methods and compositions of the disclosure may provide high drug loading (e.g., hydrogel compositions including nanoparticles that have a high drug-loading efficiency). Thus, in some embodiments, the compositions and methods of the disclosure may require lower doses and/or reduced number of applications to the eye while having a higher bioavailability.

Some embodiments described herein may provide optimum optical properties (e.g., optical transparency and wettability). In some embodiments, the compositions and methods of the disclosure may further provide patients with a drug delivery system that does not induce irritation or further inflammation or cause pain upon its application, thereby increasing patient compliance. Thus, in some embodiments, the compositions and methods of the disclosure may be well suited to be applied to the eye for extended periods of time.

Some embodiments described herein may provide a minimally invasive drug delivery vehicle and is not amenable to be removed by uncontrolled movements of the eye (e.g., uncontrolled retraction from the cornea and sclera), which could potentially cause leakage of the therapeutic agent being delivered. Thus, in some embodiments, the compositions and methods of the disclosure may provide a stable release of a therapeutic agent to a desired tissue (e.g., an optical tissue).

Some embodiments described herein may provide a composition that is biocompatible and biodegradable. For example, the compositions described herein were shown to have a high cell viability, cell adherence, and cell growth in vitro (see, e.g., Example 10). Furthermore, subcutaneous implantation of the nanoparticle-loaded hydrogel composition in rats further confirmed its in vivo biocompatibility and appropriate stability throughout 28 days see).

Some embodiments described herein may provide a composition that is easily applied to a tissue. For example, the precursor hydrogel composition loaded with nanoparticles does not require a user to mix various components. Furthermore, the precursor hydrogel composition loaded with nanoparticles does not have a limited time window (e.g., less than about 30 seconds) within which a user must apply the composition to a tissue before an undesirable change to the composition occurs (e.g., unwanted solidification, unwanted separation of components, or the like). Thus, in some embodiments, the compositions and methods of the disclosure provide a user with an efficient method to seal and deliver a therapeutic agent to a tissue.

Some embodiments described herein may provide a composition that may have a quick gelation time while retaining a high adhesive strength, especially when in contact with wet surfaces. For example, the precursor hydrogel composition loaded with nanoparticles may solidify within a few minutes (e.g., less than about 4 minutes) upon exposure to a visible light. Furthermore, the precursor hydrogel composition loaded with nanoparticles may exhibit a high adhesive strength even when in contact with a wet surface.

Some embodiments described herein may provide a composition that may have batch-to-batch reproducibility. For example, the precursor hydrogel composition loaded with nanoparticles may advantageously lack batch-to-batch product variations and the potential presence of viral contamination that is often present in naturally-derived products (e.g., that are not chemically modified). Thus, the in some embodiments, the compositions and methods of the disclosure may provide a safe and reproducible composition that is amenable to be produced at a commercial scale.

Some embodiments described herein may provide a composition that may have tunable properties. For example, the compositions described herein may be optimized at both liquid and solid states for high ocular retention upon instillation, post-crosslinking adhesion, swelling ratio, adhesive strength, and other mechanical properties while retaining high in vitro and in vivo cytocompatibility. The compositions of the disclosure may be easily optimized by varying the ratio of its components (e.g., HAGM, GelMA, photoinitiator, and nanoparticles). Thus, the in some embodiments, the compositions and methods of the disclosure may provide a flexible drug delivery platform that can be optimized for various tissue types and injuries.

### Compositions

The present disclosure features biocompatible and adhesive compositions (e.g., hydrogels) that can include one or more nanoparticles (NPs), a chemically modified gelatin (e.g., gelatin methacryloyl (GelMA)), a chemically modified hyaluronic acid (e.g., hyaluronic acid-glycidyl methacrylate (HAGM)), and a photoinitiator. In some embodiments, the compositions of the disclosure are drug-eluting hydrogels or “patches” that may facilitate the penetration of a drug through the structural barriers of tissues (e.g., ocular tissues) upon application and adhesion. For example, the compositions of the disclosure can be adhered to an ocular surface and provide a sustained release of drugs directly to an injured and/or disease site in an eye of a patient. To this end, the compositions described herein can include solubilized therapeutic agents in polymeric nanoparticles, which can further protect the therapeutic agents and help provide a sustained release of the therapeutic agent to a tissue. In some embodiments, these optimized nanoparticle formulations can be loaded within the compositions (e.g., GelPatch) of the disclosure.

Polymeric nanoparticles can be used as a drug delivery vehicle within the hydrogel compositions to deliver hydrophilic therapeutic agents. For example, polymeric nanoparticles composed of GelMA are amenable to encapsulate and deliver hydrophilic drugs. In some embodiments, the compositions described herein are a polymeric nanoparticle-based ocular drug delivery system that can achieve a sustained release of a therapeutic agent. In some embodiments, the polymeric nanoparticles are composed of GelMA and/or gelatin.

Gelatin is a derivative from collagen, which is the main structural component of the cornea. Gelatin has strong adhesive properties to cells and tissue due to the presence of RGD motifs in gelatin, a denatured form of collagen that is chemically modified to form a light-activated precursor. In some embodiments, a chemically modified gelatin can be included in the compositions of the present disclosure. In some embodiments, the chemically modified gelatin can be modified with methacryloyl anhydride (MA) to form GelMA, a photocrosslinkable derivative of gelatin. In some embodiments, chemical modification of gelatin can be performed by a synthesis reaction of gelatin with methacrylic anhydride (MAA).

In some embodiments, the nanoparticles have an aqueous, hydrophilic core. In some embodiments, the nanoparticles are biodegradable. In some embodiments, the nanoparticles are biocompatible. The nanoparticles can be formed via solvent desolvation methods, as described in Example 1, or by any other suitable methods. In some embodiments, the polymeric nanoparticles include and/or are composed of GelMA. In some embodiments, the nanoparticles include and/or are composed of GelMA having a low degree of methacrylation (e.g., about 1% to about 29%). In some embodiments, the nanoparticles include and/or are composed of GelMA having a high degree of methacrylation (e.g., about 30% to about 85%).

As described in Example 3 and Equation 1, the degree of methacrylation (DM) of GelMA can be defined as the ratio of methacrylate groups to the free amine groups in gelatin prior to the reaction. In some embodiments, the nanoparticles include and/or are composed of GelMA with a degree of methacrylation (e.g., methacryloyl functionalization) ranging from at least about 30% to about 85% (e.g., about 30% to about 65%, about 40% to about 65%, about 50% to about 65%, about 60% to about 65%, about 61% to about 65%, about 65% to about 70%, about 62% to about 68%, about 63% to about 67%, about 64% to about 66%, about 61% to about 70%, about 60% to about 75%, about 60% to about 80%, about 60% to about 85%, about 40% to about 70%, about 40% to about 75%, about 40% to about 80%, about 40% to about 85%, about 50% to about 70%, about 50% to about 75%, about 50% to about 80%, or about 50% to about 85%). In some embodiments, the nanoparticles include and/or are composed of GelMA with a degree of substitution of about 61%. In some embodiments, the GelMA includes methacrylamide substitution and methacrylate substitution. In some embodiments, the ratio of methacrylamide substitution to methacrylate substitution is between about 80:20 and 99:1. In some embodiments, the ratio of methacrylamide substitution to methacrylate substitution can range from 80:20 to 85:15, 85:25 to 90:10, 90:10 to 95:5, or 95:5 to 99:1.

In some embodiments, the concentration of GelMA in the nanoparticles can range from about 3% to about 30% (w/v) (e.g., about 3% to about 7%, about 3% to about 8%, about 30% to about 9%, about 30% to about 10%, about 30% to about 11%, about 30% to about 12%, about 3% to about 13%, about 3% to about 14%, about 3% to about 20%, about 3% to about 25%, about 3% to about 30%, about 4% to about 7%, about 4% to about 8%, about 4% to about 9%, about 4% to about 10%, about 4% to about 11%, about 4% to about 12%, about 4% to about 13%, about 4% to about 14%, about 4% to about 20%, about 4% to about 25%, about 4% to about 30%, about 5% to about 7%, about 5% to about 8%, about 5% to about 9%, about 5% to about 10%, about 5% to about 11%, about 5% to about 12%, about 5% to about 13%, about 5% to about 14%, about 5% to about 20%, about 5% to about 25%, about 5% to about 30%, about 6% to about 7%, about 6% to about 8%, about 6% to about 9%, about 6% to about 10%, about 6% to about 11%, about 6% to about 12%, about 6% to about 13%, about 6% to about 14%, about 6% to about 20%, about 6% to about 25%, about 6% to about 30%, about 7% to about 8%, about 7% to about 9%, about 7% to about 10%, about 7% to about 11%, about 7% to about 12%, about 7% to about 13%, about 7% to about 14%, about 7% to about 20%, about 7% to about 25%, about 7% to about 30%, about 15% to about 20%, about 15% to about 25%, about 15% to about 30%, about 20% to about 25%, about 20% to about 30%, or about 25% to about 30% (w/v)). In some embodiments, the nanoparticles includes GelMA at a concentration of about 10% (w/v). In some embodiments, the nanoparticles includes GelMA at a concentration of about 5% (w/v).

In some embodiments, the nanoparticles are chemically crosslinked and photo-crosslinked. In some embodiments, the nanoparticles are photo-crosslinked. In some embodiments, the nanoparticles are chemically crosslinked. In some embodiments, the nanoparticles are chemically crosslinked first and then photo-crosslinked. In some embodiments, the nanoparticles include a chemical crosslinker and a photoinitiator. In some embodiments, the use of two different types of crosslinkers (e.g., a chemical crosslinker and a photo-crosslinker) to crosslink the nanoparticles advantageously provide nanoparticles having a higher stability than the stability of nanoparticles that are only chemically crosslinked or only photo-crosslinked. As used herein, the term “nanoparticle stability” can describe the preservation of a particular nanostructure property ranging from aggregation, composition, crystallinity, shape, size, and/or surface chemistry.

In some embodiments, the nanoparticles include glutaraldehyde as a chemical crosslinker. When added to the GelMA precursor nanoparticle solution, the reaction between the amine groups of GelMA and the carbonyl groups of glutaraldehyde leads to the formation of a GelMA hydrogel network (e.g., the shell of the nanoparticles) incorporated with the glutaraldehyde cross-linker molecule.

In some embodiments, the nanoparticles include a chemical crosslinker (e.g., glutaraldehyde) at a concentration of about 0% to about 1% (w/v) (e.g., about 0% to about 0.1%, about 0% to about 0.2%, about 0% to about 0.3%, about 0% to about 0.4%, about 0% to about 0.5%, about 0% to about 0.6%, about 0% to about 0.7%, about 0% to about 0.8%, about 0% to about 0.9%, about 0.1% to about 0.2%, about 0.1% to about 0.3%, about 0.1% to about 0.4%, about 0.1% to about 0.5%, about 0.1% to about 0.6%, about 0.1% to about 0.7%, about 0.1% to about 0.8%, about 0.1% to about 0.9%, about 0.1% to about 1%, about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, about 0.2% to about 1%, about 0.3% to about 0.4%, about 0.3% to about 0.5%, about 0.3% to about 0.6%, about 0.3% to about 0.7%, about 0.3% to about 0.8%, about 0.3% to about 0.9%, about 0.3% to about 1%, about 0.4% to about 0.5%, about 0.4% to about 0.6%, about 0.4% to about 0.7%, about 0.4% to about 0.8%, about 0.4% to about 0.9%, about 0.4% to about 1%, about 0.5% to about 0.6%, about 0.5% to about 0.7%, about 0.5% to about 0.8%, about 0.5% to about 0.9%, about 0.5% to about 1%, about 0.6% to about 0.7%, about 0.6% to about 0.8%, about 0.6% to about 0.9%, about 0.6% to about 1%, about 0.7% to about 0.8%, about 0.7% to about 0.9%, about 0.7% to about 1%, about 0.8% to about 0.9%, about 0.8% to about 1%, or about 0.9% to about 1%). In some embodiments, the nanoparticles include glutaraldehyde at a concentration of about 0.4% (w/v). In some embodiments, the nanoparticles include glutaraldehyde at a concentration of about 0% (w/v). In some embodiments, the nanoparticles include glutaraldehyde at a concentration of about 0.2% (w/v). In some embodiments, the nanoparticles include glutaraldehyde at a concentration of about 0.8% (w/v).

Non-limiting examples of chemical crosslinkers that can be used to chemically crosslink the nanoparticles include 1-ethyl-3-(3-dimethylaminopropyl) carbodiimide hydrochloride (EDC)—N-Hydroxysuccinimide (NHS) coupling, which forms amide bonds between existing carboxyl and amine groups of polymer chains or via formation of thioether bonds and a maleimide-functionalized polymer, which can interact with existing thiol groups in the polymer backbone (e.g., cysteine).

In some embodiments, the nanoparticles can include a photoinitiator that can be used to further activate polymerization and solidification of the nanoparticles when they are in a precursor or non-solid (e.g., viscous liquid or gel) form. In some embodiments, exposing the precursor nanoparticle composition to light activates the photoinitiator, triggering the formation of free-radicals, resulting in vinyl-bond crosslinking between methacrylate groups, and thus polymerization of the composition, which results in a physical change of the precursor nanoparticle composition from an only chemically crosslinked nanoparticle composition to a chemically crosslinked and photo-crosslinked nanoparticle composition.

Different types of light sources can be used to photo-crosslink the nanoparticle precursor composition (e.g., the composition in a solution form). Non-limiting examples of light sources that can be used to polymerize the nanoparticle precursor composition include visible light sources (e.g., white or blue light), ultraviolet (UV) light sources, near-infrared light sources, and fluorescent light sources. In some embodiments, the nanoparticle precursor composition includes a UV light-activated photoinitiator. In some embodiments, the nanoparticle precursor composition is photo-crosslinked by activating the UV light-activated photoinitiator upon exposure of light having a wavelength between about 100 nanometers (nm) to about 400 nm (e.g., about 100 nm to about 200 nm, about 100 nm to about 280 nm, about 100 nm to about 320 nm, about 100 nm to about 380 nm, about 200 nm to about 280 nm, about 200 nm to about 320 nm, about 200 nm to about 380 nm, about 200 nm to about 400 nm, about 280 nm to about 320 nm, about 280 nm to about 380 nm, about 280 nm to about 400 nm, about 300 nm to about 400 nm, about 300 nm to about 365 nm, about 365 nm to about 400 nm, about 320 nm to about 380 nm, about 320 nm to about 400 nm, or about 380 nm to about 400 nm). In some embodiments, the UV light-activated photoinitiator can be activated upon exposure of light having a wavelength of about 320 nm. In some embodiments, the UV light-activated photoinitiator can be activated upon exposure of light having a wavelength of about 365 nm. In some embodiments, the UV light-activated photoinitiator can be activated upon exposure of light having a wavelength of about 380 nm. In some embodiments, the UV light-activated photoinitiator can be activated upon exposure of light having a wavelength of about 400 nm.

In some embodiment, the UV light-activated photoinitiator includes 2-hydroxy-4′-(2-hydroxyethoxy)-2-methylpropiophenone (i.e., Irgacure® 2959). In some embodiment, the UV light-activated photoinitiator includes 2-hydroxy-2-methyl propiophenone (Irgacure® 1173). In some embodiment, the UV light-activated photoinitiator includes diphenyl(2,4,6-trimethylbenzoyl)phosphine oxide (Irgacure® TPO). In some embodiment, the UV light-activated photoinitiator includes 1-hydroxycyclohexyl phenyl ketone (Irgacure® 184). In some embodiment, the UV light-activated photoinitiator includes 2-benzyl-2-(dimethylamino)-1-[4-(morpholinyl) phenyl)]-1-butanone (Irgacure® 369). In some embodiment, the UV light-activated photoinitiator includes 2,2-dimethoxy-2-phenyl acetophenone (Irgacure® 651). In some embodiment, the UV light-activated photoinitiator includes 2-methyl-4′-(methylthio)-2-morpholinopropiophenone (Irgacure® 907).

Skilled practitioners will appreciate that any number of known materials can be used to prepare nanoparticles, including, but not limited to, biodegradable and biocompatible polymers. In some embodiments, the nanoparticles include and/or are composed of GelMA and HAGM. Additional polymers that can be used to generate the nanoparticles to be dispersed in the composition include, but are not limited to poly(ethylene) glycol, poly(vinyl) pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate polycaprolactone, cellulose, alginate, chitosan, starch, or any combination thereof.

In some embodiments, the composition includes nanoparticles having a diameter ranging from about 130 nm to about 350 nm or more (e.g., about 130 nm to about 140 nm, about 130 nm to about 145 nm, about 130 nm to about 150 nm, about 130 nm to about 160 nm, about 130 nm to about 200 nm, about 130 nm to about 235 nm, about 130 nm to about 250 nm, about 130 nm to about 285 nm, about 130 nm to about 300 nm, about 130 nm to about 350 nm, about 140 nm to about 145 nm, about 140 nm to about 150 nm, about 140 nm to about 160 nm, about 140 nm to about 200 nm, about 140 nm to about 235 nm, about 140 nm to about 250 nm, about 140 nm to about 285 nm, about 140 nm to about 300 nm, about 140 nm to about 350 nm, about 145 nm to about 150 nm, about 145 nm to about 160 nm, about 145 nm to about 200 nm, about 145 nm to about 235 nm, about 145 nm to about 250 nm, about 145 nm to about 285 nm, about 145 nm to about 300 nm, about 145 nm to about 350 nm, about 150 nm to about 160 nm, about 150 nm to about 200 nm, about 150 nm to about 235 nm, about 150 nm to about 250 nm, about 150 nm to about 285 nm, about 150 nm to about 300 nm, about 150 nm to about 350 nm, about 160 nm to about 200 nm, about 160 nm to about 235 nm, about 160 nm to about 250 nm, about 160 nm to about 285 nm, about 160 nm to about 300 nm, about 160 nm to about 350 nm, about 200 nm to about 235 nm, about 200 nm to about 250 nm, about 200 nm to about 285 nm, about 200 nm to about 300 nm, about 200 nm to about 350 nm, about 235 nm to about 250 nm, about 235 nm to about 285 nm, about 235 nm to about 300 nm, about 235 nm to about 350 nm, about 250 nm to about 285 nm, about 250 nm to about 300 nm, about 250 nm to about 350 nm, about 285 nm to about 300 nm, about 285 nm to about 350 nm, or about 300 nm to about 350 nm). In some embodiments, the nanoparticles have a diameter of about 200 nm. In some embodiments, the nanoparticles have a diameter of about 160 nm. In some embodiments, the nanoparticles have a diameter of about 285 nm. In some embodiments, the nanoparticles have a diameter of about 235 nm.

The polydispersity index (PDI) is a measure of the heterogeneity of a sample based on size. For example, the PDI describes the width or spread of a particle size distribution. The PDI value is dimensionless and may vary from 0 to 1, where particles (e.g., micelles) with PDI values approaching 0 (e.g., less than about 0.1) may imply a monodisperse particle size distribution, and particles (e.g., micelles) with PDI values approaching 1 may imply a more polydisperse particle size distribution. In some embodiments, the composition includes nanoparticles having a polydispersity index ranging from about at least 0.01 to about 0.2 (e.g., about 0.01 to about 0.08, about 0.01 to about 0.9, about 0.01 to about 0.1, about 0.01 to about 0.2, about 0.02 to about 0.08, about 0.02 to about 0.9, about 0.02 to about 0.1, about 0.02 to about 0.2, about 0.03 to about 0.08, about 0.03 to about 0.9, about 0.03 to about 0.1, about 0.03 to about 0.2, about 0.04 to about 0.08, about 0.04 to about 0.9, about 0.04 to about 0.1, about 0.04 to about 0.2, about 0.05 to about 0.08, about 0.05 to about 0.9, about 0.05 to about 0.1, about 0.05 to about 0.2, about 0.06 to about 0.08, about 0.06 to about 0.9, about 0.06 to about 0.1, about 0.06 to about 0.2, about 0.07 to about 0.08, about 0.07 to about 0.9, about 0.07 to about 0.1, about 0.07 to about 0.2, about 0.08 to about 0.9, about 0.08 to about 0.1, about 0.08 to about 0.2, about 0.09 to about 0.1, about 0.09 to about 0.2, or about 0.1 to about 0.2). In some embodiments, the nanoparticles have a polydispersity index of about 0.2 at most.

In some embodiments, the composition includes nanoparticles having a negative surface charge density. In some embodiments, the composition includes nanoparticles having a surface charge of about −4 millivolts (mV) to about −6 mV (e.g., about −4 mV to about −4.5 mV, about −4 mV to about −5 mV, about −4 mV to about −5.5 mV, about −4 mV to about −6 mV, about −4.5 mV to about −5 mV, about −4.5 mV to about −5.5 mV, about −4.5 mV to about −6 mV, about −5 mV to about −5.5 mV, about −5 mV to about −6 mV, or about −5.5 mV to about −6 mV). In some embodiments, the nanoparticles have a surface charge of about −5 mV.

In some embodiments, the composition includes nanoparticles at a concentration of about 5 mg/ml to about 50 mg/ml (e.g., about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 35 mg/ml, about 5 mg/ml to about 40 mg/ml, about 5 mg/ml to about 45 mg/ml, about 5 mg/ml to about 49 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 35 mg/ml, about 10 mg/ml to about 40 mg/ml, about 10 mg/ml to about 45 mg/ml, about 10 mg/ml to about 50 mg/ml, about 15 mg/ml to about 20 mg/ml, about 15 mg/ml to about 25 mg/ml, about 15 mg/ml to about 30 mg/ml, about 15 mg/ml to about 35 mg/ml, about 15 mg/ml to about 40 mg/ml, about 15 mg/ml to about 45 mg/ml, about 15 mg/ml to about 50 mg/ml, about 20 mg/ml to about 25 mg/ml, about 20 mg/ml to about 30 mg/ml, about 20 mg/ml to about 35 mg/ml, about 20 mg/ml to about 40 mg/ml, about 20 mg/ml to about 45 mg/ml, about 20 mg/ml to about 50 mg/ml, about 25 mg/ml to about 30 mg/ml, about 25 mg/ml to about 35 mg/ml, about 25 mg/ml to about 40 mg/ml, about 25 mg/ml to about 45 mg/ml, about 25 mg/ml to about 50 mg/ml, about 30 mg/ml to about 35 mg/ml, about 30 mg/ml to about 40 mg/ml, about 30 mg/ml to about 45 mg/ml, about 30 mg/ml to about 50 mg/ml, about 35 mg/ml to about 40 mg/ml, about 35 mg/ml to about 45 mg/ml, about 35 mg/ml to about 50 mg/ml, about 40 mg/ml to about 45 mg/ml, about 40 mg/ml to about 50 mg/ml, or about 45 mg/ml to about 50 mg/ml).

In some embodiments, the compositions described herein include one or more therapeutic agents (e.g., as a drug delivery payload). In some embodiments, the nanoparticles include one or more therapeutic agents. In some embodiments, the therapeutic agents are encapsulated in, carried by, or otherwise loaded in or on the nanoparticles and are not free within the composition (e.g., external to the nanoparticles). In some embodiments, one or more therapeutic agents are dispersed within the composition but not loaded in or on the nanoparticles. In other words, the therapeutic agents may be dispersed within the composition external to the nanoparticles. In some embodiments, a first therapeutic agent is encapsulated in, carried by, or otherwise loaded in or on the nanoparticles, and a second therapeutic agent is outside the nanoparticles within the same composition. In some embodiments, the first and second therapeutic agents are the same. In some embodiments, the first and second therapeutic agents are the different. In some embodiments, the therapeutic agents are dispersed, embedded, suspended, and/or mixed within the composition.

In some embodiments, the nanoparticles encapsulate a therapeutic agent at an encapsulation efficiency ranging from at least about 55% to about 100% (e.g., about 55% to about 60%, about 55% to about 65%, about 55% to about 70%, about 55% to about 75%, about 55% to about 80%, about 55% to about 85%, about 55% to about 90%, about 55% to about 95%, about 55% to about 100%, about 60% to about 65%, about 60% to about 70%, about 60% to about 75%, about 60% to about 80%, about 60% to about 85%, about 60% to about 90%, about 60% to about 95%, about 60% to about 100%, about 85% to about 90%, about 85% to about 95%, about 85% to about 100%, about 90% to about 95%, about 90% to about 100%, or about 95% to about 100%). In some embodiments, the nanoparticles encapsulate a therapeutic agent at an encapsulation efficiency of about 94%.

In some embodiments, the amount of therapeutic agent loaded inside the nanoparticles varies with an initial concentration of the therapeutic agent during the loading phase (e.g., during the synthesis of the nanoparticles). In some embodiments, the nanoparticles achieve a maximum loading capacity at a therapeutic agent concentration of about 0.01% (w/v) to about 1.5% (w/v) (e.g., about 0.01% to about 0.05%, about 0.01% to about 0.1%, about 0.01% to about 0.2%, about 0.01% to about 0.25%, about 0.01% to about 0.3%, about 0.01% to about 0.4%, about 0.01% to about 0.5%, about 0.01% to about 0.6%, about 0.01% to about 0.7%, about 0.01% to about 0.8%, about 0.01% to about 0.9%, about 0.01% to about 1%, about 0.05% to about 0.1%, about 0.05% to about 0.2%, about 0.05% to about 0.25%, about 0.05% to about 0.3%, about 0.05% to about 0.4%, about 0.05% to about 0.5%, about 0.05% to about 0.6%, about 0.05% to about 0.7%, about 0.05% to about 0.8%, about 0.05% to about 0.9%, about 0.05% to about 1%, about 0.25% to about 0.3%, about 0.25% to about 0.4%, about 0.25% to about 0.5%, about 0.25% to about 0.6%, about 0.25% to about 0.7%, about 0.25% to about 0.8%, about 0.25% to about 0.9%, about 0.25% to about 1%, about 0.5% to about 0.6%, about 0.5% to about 0.7%, about 0.5% to about 0.8%, about 0.5% to about 0.9%, or about 0.5% to about 1%). In some embodiments, the nanoparticles achieve a maximum loading capacity at a therapeutic agent concentration of about 0.05% (w/v). In some embodiments, the nanoparticles achieve a maximum loading capacity at a therapeutic agent concentration of about 0.25% (w/v). In some embodiments, the nanoparticles achieve a maximum loading capacity at a therapeutic agent concentration of about 0.5% (w/v). In some embodiments, the nanoparticles achieve a maximum loading capacity at a therapeutic agent concentration of about 1% (w/v).

In some embodiments, the therapeutic agent is present in the composition at a concentration of about 0.25 μg/mL to about 5 mg/mL (e.g., about 0.25 μg/mL to about 0.5 μg/mL, about 0.25 μg/mL to about 0.75 μg/mL, about 0.25 μg/mL to about 1 μg/mL, about 0.25 μg/mL to about 10 μg/mL, about 0.25 μg/mL to about 100 μg/mL, about 0.25 μg/mL to about 250 μg/mL, about 0.25 μg/mL to about 500 μg/mL, about 0.25 μg/mL to about 750 μg/mL, about 0.25 μg/mL to about 1 mg/mL, about 0.25 μg/mL to about 2 mg/mL, about 0.25 μg/mL to about 3 mg/mL, about 0.25 μg/mL to about 4 mg/mL, about 0.25 μg/mL to about 5 mg/mL, about 1 μg/mL to about 10 μg/mL, about 1 μg/mL to about 100 μg/mL, about 1 μg/mL to about 250 μg/mL, about 1 μg/mL to about 500 μg/mL, about 1 μg/mL to about 750 μg/mL, about 1 μg/mL to about 1 mg/mL, about 1 μg/mL to about 2 mg/mL, about 1 μg/mL to about 3 mg/mL, about 1 μg/mL to about 4 mg/mL, about 1 μg/mL to about 5 mg/mL, about 10 μg/mL to about 100 μg/mL, about 10 μg/mL to about 250 μg/mL, about 10 μg/mL to about 500 μg/mL, about 10 μg/mL to about 750 μg/mL, about 10 μg/mL to about 1 mg/mL, about 10 μg/mL to about 2 mg/mL, about 10 μg/mL to about 3 mg/mL, about 10 μg/mL to about 4 mg/mL, about 10 μg/mL to about 5 mg/mL, about 100 μg/mL to about 250 μg/mL, about 100 μg/mL to about 500 μg/mL, about 100 μg/mL to about 750 μg/mL, about 100 μg/mL to about 1 mg/mL, about 100 μg/mL to about 2 mg/mL, about 100 μg/mL to about 3 mg/mL, about 100 μg/mL to about 4 mg/mL, about 100 μg/mL to about 5 mg/mL, about 250 μg/mL to about 500 μg/mL, about 250 μg/mL to about 750 μg/mL, about 250 μg/mL to about 1 mg/mL, about 250 μg/mL to about 2 mg/mL, about 250 μg/mL to about 3 mg/mL, about 250 μg/mL to about 4 mg/mL, about 250 μg/mL to about 5 mg/mL, about 500 μg/mL to about 750 μg/mL, about 500 μg/mL to about 1 mg/mL, about 500 μg/mL to about 2 mg/mL, about 500 μg/mL to about 3 mg/mL, about 500 μg/mL to about 4 mg/mL, about 500 μg/mL to about 5 mg/mL, about 750 μg/mL to about 1 mg/mL, about 750 μg/mL to about 2 mg/mL, about 750 μg/mL to about 3 mg/mL, about 750 μg/mL to about 4 mg/mL, about 750 μg/mL to about 5 mg/mL, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, or about 3 mg/mL to about 5 mg/mL). In some embodiments, the therapeutic agent is present in the composition at a concentration of about 3 mg/mL. In some embodiments, the therapeutic agent is present in the composition at a concentration that is dependent upon the potency of applied therapeutic.

In some embodiments, the therapeutic agent is a hydrophobic therapeutic agent, a hydrophilic therapeutic agent, an amphiphilic therapeutic agent, or any combination thereof. In some embodiments, the therapeutic agent is a hydrophilic therapeutic agent. In some embodiments, the therapeutic agent is an antibacterial agent. In some embodiments, the therapeutic agent is an antibiotic drug. In some embodiments, the therapeutic agent is moxifloxacin (MXF). In some embodiments, the therapeutic agent includes antibiotic drugs, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, chitosan, or any combination thereof.

Other exemplary therapeutic agents for inclusion in the compositions include, but are not limited to, an anti-fungal, an anti-viral, an anti-acanthamoebal, an anti-inflammatory, an immunosuppressive, an anti-glaucoma, an anti-VEGF, a growth factor, or any combination thereof.

In some embodiments, a composition, which includes a therapeutic agent encapsulated in the nanoparticles, has improved healing properties compared to a composition without a therapeutic agent (e.g., it can reduce the time that it takes for an ocular injury to heal when treated with the composition, as compared to treatment with other commercially available ocular sealants or to compositions that do not include a therapeutic agent, for example). In some embodiments, a therapeutic agent released by the nanoparticles in the composition can reduce the risk of inflammation and infection following injury. In some embodiments, the therapeutic agent released by the nanoparticles in the composition can promote wound healing in an injured tissue (e.g., an injured optical tissue).

Non-limiting examples of suitable anti-inflammatory agents include a steroidal anti-inflammatory drug (e.g., prednisolone), a non-steroidal anti-inflammatory drug (e.g., bromfenac), an mTOR inhibitor, a calcineurin inhibitor, a synthetic or natural anti-inflammatory protein, antiproliferative drugs (e.g., dexamethasone, 5-fluorouracil, daunomycin, paclitaxel, curcumin, resveratrol, and mitomycin), methylprednisolone, prednisolone, hydrocortisone, fludrocortisone, prednisone, celecoxib, ketorolac, piroxicam, diclorofenac, ibuprofen, and ketoprofen, rapamycin, cyclosporin, and tacrolimus/FK-506.

In some embodiments, the growth factor is epithelial growth factor, fibroblast growth factor, nerve growth factor, hepatocyte growth factor, or any combination thereof. Further non-limiting examples of suitable growth factors include transforming growth factors (TGFs) (e.g., beta transforming growth factors such as, TGF-01, TGF-02, TGF-03), fibroblast growth factors (FGFs), platelet derived growth factors (PDGFs), epidermal growth factors (EGFs), connective tissue activated peptides (CTAPs), osteogenic factors, bone morphogenetic proteins (e.g., BMP-1, BMP-2, BMP-3, BMP-4, BMP-5, BMP-6, BMP-7, BMP-8, BMP-9); heparin-binding growth factors (e.g., fibroblast growth factor (FGF), epidermal growth factor (EGF), insulin-like growth factor (IGF)), Inhibins (e.g., Inhibin A, Inhibin B), growth differentiating factors (for example, GDF-1), and Activins (e.g., Activin A, Activin B, Activin AB), and biologically active analogs, fragments, and derivatives of such growth factors.

In addition to the nanoparticles, the compositions of the disclosure can include gelatin methacryloyl (GelMA), hyaluronic acid-glycidyl methacrylate (HAGM), and a visible light-activated photoinitiator. In some embodiments, the composition is a hydrogel having nanoparticles suspended within its three-dimensional matrix. In some embodiments, the physical properties of hydrogels may be similar to native tissue, and hydrogels may be used to encapsulate therapeutic agents in the hydrogel matrix formed upon gelation. In some embodiments, the composition is an injectable hydrogel, which may be injected into a subject in need thereof.

In some embodiments, at least two of the GelMA, the HAGM, the visible light-activated photoinitiator, and the one or more nanoparticles are formulated in separate formulations. For example, in some embodiments, the nanoparticles are formulated separately from the precursor hydrogel composition including GelMA, HAGM, and the visible light-activated photoinitiator. In some embodiments, the nanoparticles are first formulated to encapsulate a therapeutic agent and then are mixed with the precursor hydrogel composition including GelMA, HAGM, and the visible light-activated photoinitiator.

Generally, a hydrogel may be formed by using at least one, or one or more types of hydrogel precursors, and setting or solidifying the one or more types of hydrogel precursors in an aqueous solution to form a three-dimensional network, wherein formation of the three-dimensional network may cause the one or more types of hydrogel precursors to gel. As used herein, the term “hydrogel precursor” refers to any chemical compound that may be used to form a hydrogel. Examples of hydrogel precursors include, but are not limited to, a natural polymer, a hydrophilic monomer, a hydrophilic polymer, a hydrophilic copolymer formed from a monomer and a polymer. In some embodiments, the hydrogel precursor includes a chemically-modified polymer. The chemically-modified polymer may form a three-dimensional network in an aqueous medium to form a hydrogel. In some embodiments, the chemically-modified polymer is GelMA and/or HAGM.

Hyaluronic acid (HA) is a viscoelastic and highly biocompatible glycosaminoglycan, that is naturally present in the cornea. HA is known to play a role in the regeneration and reconstruction of soft tissues. In some embodiments, a chemically modified HA can be included in the compositions of the present disclosure. In some embodiments, the chemically modified HA can be methacrylated hyaluronic acid or a photocrosslinkable derivative of HA. In some embodiments, methacrylation of HA can be performed by ring opening of the HA backbone reaction and a reversible transesterification reaction. In some embodiments, the methacrylated hyaluronic acid included in the composition is HAGM.

In some embodiments, HAGM is present in the composition at a concentration of about 0.5% and about 5% weight per volume (w/v) (e.g., about 0.5% to about 5%, about 1% to about 5%, about 1.5% to about 5%, about 2% to about 5%, about 2.5% to about 5%, about 3% to about 5%, about 0.5% to about 3%, about 0.5% to about 4%, about 1% to about 3%, or about 2% to about 3% (w/v)). In some embodiments, the HAGM is present in the composition at a concentration 3% (w/v). In some embodiments, the HAGM is present in the composition at a concentration 0.5% (w/v).

In some embodiments, the composition includes GelMA with a degree of methacrylation (i.e., methacryloyl functionalization) ranging from at least about 30% to about 85% (e.g., about 30% to about 65%, about 40% to about 65%, about 50% to about 65%, about 60% to about 65%, about 61% to about 65%, about 65% to about 70%, about 62% to about 68%, about 63% to about 67%, about 64% to about 66%, about 61% to about 70%, about 60% to about 75%, about 60% to about 80%, about 60% to about 85%, about 40% to about 70%, about 40% to about 75%, about 40% to about 80%, about 40% to about 85%, about 50% to about 70%, about 50% to about 75%, about 50% to about 80%, or about 50% to about 85%). In some embodiments, the composition includes GelMA with a degree of substitution of about 61%. In some embodiments, the GelMA includes methacrylamide substitution and methacrylate substitution. In some embodiments, the ratio of methacrylamide substitution to methacrylate substitution is between about 80:20 and 99:1. In some embodiments, the ratio of methacrylamide substitution to methacrylate substitution can range from 80:20 to 85:15, 85:25 to 90:10, 90:10 to 95:5, or 95:5 to 99:1.

In some embodiments, the concentration of GelMA in the composition can range from about 3% to about 14% (w/v) (e.g., about 3% to about 7%, about 3% to about 8%, about 30% to about 9%, about 30% to about 10%, about 30% to about 11%, about 30% to about 12%, about 3% to about 13%, about 3% to about 14%, about 4% to about 7%, about 4% to about 8%, about 4% to about 9%, about 4% to about 10%, about 4% to about 11%, about 4% to about 12%, about 4% to about 13%, about 4% to about 14%, about 5% to about 7%, about 5% to about 8%, about 5% to about 9%, about 5% to about 10%, about 5% to about 11%, about 5% to about 12%, about 5% to about 13%, about 5% to about 14%, about 6% to about 7%, about 6% to about 8%, about 6% to about 9%, about 6% to about 10%, about 6% to about 11%, about 6% to about 12%, about 6% to about 13%, about 6% to about 14%, about 7% to about 8%, about 7% to about 9%, about 7% to about 10%, about 7% to about 11%, about 7% to about 12%, about 7% to about 13%, or about 7% to about 14% (w/v)). In some embodiments, the composition includes GelMA at a concentration of about 7% (w/v). In some embodiments, the composition includes one or more polymeric micelles, GelMA at a concentration of about 7% (w/v), and HAGM at a concentration of about 3% (w/v).

In some embodiments, the compositions can include a photoinitiator that can be used to activate polymerization and solidification of the composition when it is in a non-solid (e.g., viscous liquid, gel, liquid, or solution) form. In some embodiments, exposing the composition to light activates the photoinitiator, triggering the formation of free-radicals, resulting in vinyl-bond crosslinking between methacrylate groups, and thus polymerization of the composition, which results in a physical change of the composition from a solvent form to a hydrogel form. In some embodiments, the composition is in a form of a solution. In some embodiments, the composition is in a form of a hydrogel.

Different types of light sources can be used to photo-crosslink the hydrogel precursor composition (e.g., the composition in a solution form). Non-limiting examples of light sources that can be used to polymerize the composition include visible light sources (e.g., white or blue light), ultraviolet light sources, near-infrared light sources, and fluorescent light sources. In some embodiments, the composition includes a visible light-activated photoinitiator that can be activated upon exposure of light having a wavelength between about 420 nanometers (nm) to 550 nm. In some embodiments, the visible light-activated photoinitiator can be activated upon exposure of light having a wavelength of about 460 nm. In some embodiments, the visible light-activated photoinitiator can be activated upon exposure of light having a wavelength ranging from about 400 nm to about 800 nm. In some embodiments, the visible light-activated photoinitiator can be activated upon exposure of light having a wavelength less than 800, 750, 700, 650, 600, 550, 500, 450, or 400 nm. In some embodiments, the visible light-activated photoinitiator can be activated upon exposure of light having a wavelength greater than 400, 450, 500, 550, or 600 nm.

In some embodiments, the photoinitiator includes a light-activated photoinitiator. In some embodiments, the photoinitiator is. In some embodiment, the light-activated photoinitiator includes an ultraviolet light-activated photoinitiator. In some embodiment, the light-activated photoinitiator includes a near-infrared (NIR) light-activated photoinitiator. In some embodiment, the light-activated photoinitiator includes a visible light-activated photoinitiator. In some embodiment, the visible light-activated photoinitiator includes Eosin Y, triethanolamine (TEA), N-vinylcaprolactam (VC), or any combination thereof. In some embodiments, the light-activated photoinitiator can includes a blue light-activated photoinitiator. In some embodiments, the visible light-activated photoinitiator includes triethanolamine, N-vinylcaprolactam, riboflavin, 2-hydroxy-4′-(2-hydroxyethoxy)-2-methylpropiophenone, Eosin Y disodium salt, 4,6-trimethylbenzoylphosphinate, triethanol amine, dl-2,3-diketo-1,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide, 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane, derivatives thereof, or any combination thereof. In some embodiments, the visible light-activated photoinitiator includes a mixture of triethanolamine, N-vinylcaprolactam, riboflavin, 2-hydroxy-4′-(2-hydroxyethoxy)-2-methylpropiophenone, and Eosin Y disodium salt. In some embodiments, the visible light-activated photoinitiator comprises a mixture of two or more elements selected from triethanolamine, N-vinylcaprolactam, riboflavin, 2-hydroxy-4′-(2-hydroxyethoxy)-2-methylpropiophenone, and Eosin Y disodium salt.

The release kinetics of the composition including nanoparticles loaded with a therapeutic agent can be controlled by adjusting the concentration of one or more of the polymers in the formulation (e.g., HAGM and/or GelMA), the chemical crosslinker, and/or the photoinitiator. In some embodiments, the composition delivers at least about 50% to about 100% of the therapeutic agent from the micelles in about 5 days. In some embodiments, the composition exhibits a burst release within about 2 hours after application. In some embodiments, about 20% to about 50% (e.g., about 20% to about 30%, about 20% to about 40%, about 20% to about 45%, about 20% to about 50%, or more) of the therapeutic agent is released from the nanoparticles and the composition within about 2 hours after coming in contact with an aqueous solution. In some embodiments, about 30% of the therapeutic agent is released from the nanoparticles and the composition within about 2 hours after coming in contact with an aqueous solution. In some embodiments, about 35% of the therapeutic agent is released from the nanoparticles and the composition within about 2 hours after coming in contact with an aqueous solution. In some embodiments, about 40% of the therapeutic agent is released from the nanoparticles and the composition within about 2 hours after coming in contact with an aqueous solution. In some embodiments, about 45% of the therapeutic agent is released from the nanoparticles and the composition within about 2 hours after coming in contact with an aqueous solution.

The incorporation of nanoparticles within the hydrogel composition can enable loading of hydrophilic therapeutic agents into the hydrogel composition and its sustained release within an extended time frame (e.g., about 5 days). In some embodiments, the incorporation of nanoparticles into the hydrogel composition retards the release of the therapeutic agent and leads to a sustained drug release profile over an extended time frame (e.g., about 5 days to about 15 days) after an initial burst release. In some embodiments, the composition has a sustained therapeutic release profile over a period of about 15 days at most. In some embodiments, about 95% to about 100% (e.g., about 99%) of the therapeutic agent is released within about 5 to about 15 days.

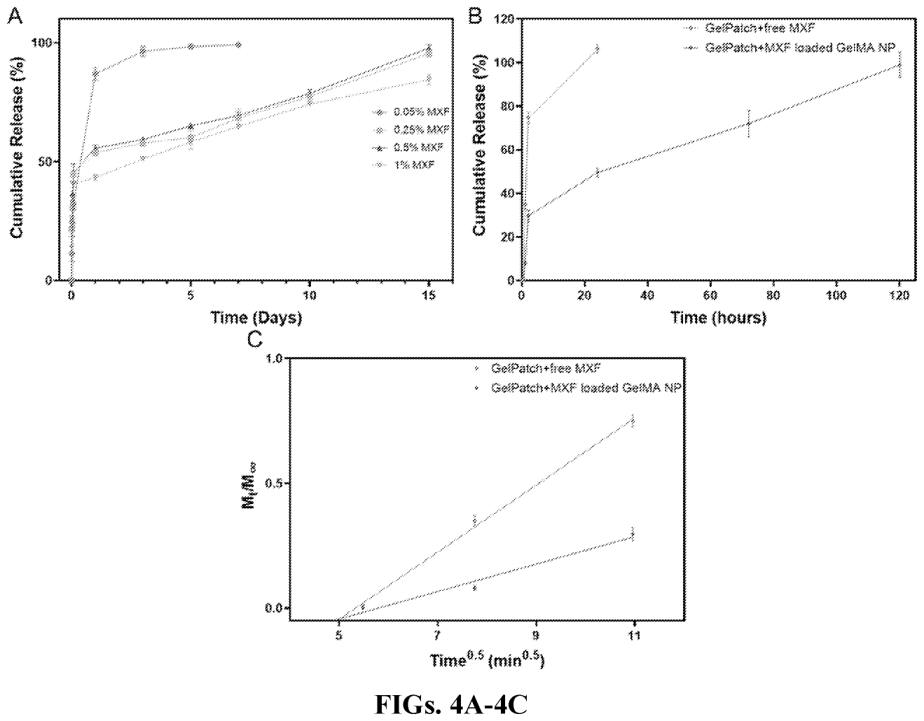

As shown in FIG. 4C and Table 1 and as disclosed in Example 3, the diffusion coefficient D of the composition can be calculated based on Fick's second law and the slope of plotted drug release data. In some embodiments, the diffusion coefficient D of the composition ranges from about 2.5×10⁻¹⁰ m²·s⁻¹ to about 3.5×10⁻¹⁰ m²·s⁻¹ (e.g., about 2.5×10⁻¹⁰ m²·s⁻¹ to about 3×10⁻¹⁰ m²·s⁻¹ or 3×10⁻¹⁰ m²·s⁻¹ to about 3.5×10⁻¹⁰ m²·s⁻¹).

In some embodiments, the drug release profile can be controlled by controlling the density of crosslinking, which can have impact on release rate of a payload (e.g., small drug molecules). In some embodiments, the drug release profile can be decreased by increasing the density of crosslinking of the polymer(s) of the nanoparticle and/or hydrogel compositions. In some embodiments, increasing the density of crosslinking can be controlled by increasing the photo-crosslinking time, increasing the photoinitiator composition, changing the GelMA/HAGM concentration, or any combination thereof. In some embodiments, since the release of small molecules can be controlled via diffusion, the higher the crosslinking density, the more steric hindrance and the slower the release of the small molecule from the any of the disclosed compositions.

In some embodiments, the drug release profile can be decreased by increasing the stiffness of crosslinking of the polymer(s) of the nanoparticle and/or hydrogel compositions. Alternatively, in some embodiments, the drug release profile can be increased by decreasing the stiffness of crosslinking of the polymer(s) of the nanoparticle and/or hydrogel compositions. In some embodiments, the drug release profile can be decreased by decreasing the swelling properties of the polymer(s) of the nanoparticle and/or hydrogel compositions. Alternatively, in some embodiments, the drug release profile can be increased by increasing the swelling properties of the polymer(s) of the nanoparticle and/or hydrogel compositions.

In some embodiments, the drug release profile can be controlled by the molecular structure of the polymer(s) of the nanoparticle and/or hydrogel compositions. For example, in some embodiments, some blocks of polymer and/or protein chains of the nanoparticle and/or hydrogel compositions can form hydrophobic interactions with the payload (e.g., a drug molecule) and slow down the payload release.

In some embodiments, the drug release profile (e.g., of a hydrophobic drug molecule payload) can be controlled by the concentration of the polymer(s) of the nanoparticle and/or hydrogel compositions due to the formation of a denser crosslinked matrix. For example, in some embodiments, a hydrogel and/or nanoparticle composition having an increased polymeric concentration(s) can yield a denser, crosslinked matrix, thereby causing a slower drug release profile (e.g., of a hydrophobic drug molecule payload).

In some embodiments, the drug release profile can be controlled by the molecular structure of the payload (e.g., a small molecule, a small drug molecule, a hydrophilic drug molecule, a hydrophobic drug molecule, or the like) itself. For example, in some embodiments, the payload can be an enantiomer, which may have the tendency to form a crystalline structure more than an amorphous structure, which can significantly impact its release profile. For example, in some embodiments, the payload having a crystalline structure may have a slower release profile than the payload having an amorphous structure. In some embodiments, the formulation process of the payload (e.g., a small molecule, a small drug molecule, a hydrophilic drug molecule, a hydrophobic drug molecule, or the like) can induce crystallization of the loaded drug molecules, thereby slowing the drug release profile. In some embodiments, the concentration (e.g., a crystallization concentration) of the payload (e.g., a small molecule, a small drug molecule, a hydrophilic drug molecule, a hydrophobic drug molecule, or the like) can induce crystallization of the loaded drug molecules, thereby slowing the drug release profile. In some embodiments, if the payload is a hydrophilic drug molecule, the drug release profile can be faster (e.g., due to faster diffusion out of the nanoparticle and/or hydrogel compositions). In some embodiments, the drug release profile can be slowed down by introducing larger interfaces (e.g., by increasing the diameter of the nanoparticles loaded with the payload, such as a drug molecule).

In some embodiments, the composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH-buffered solution (e.g. PBS).

In some embodiments, the pharmaceutically acceptable carrier is a topical carrier. In some embodiments, the composition is formulated for topical use. In some embodiments, the composition is topically administered to a tissue (e.g., an ocular tissue) of a patient. In some embodiments, the composition can be applied to a tissue (e.g., an ocular tissue) for topical, targeted delivery of a therapeutic agent.

### Physical Properties of Compositions

The physical properties of the compositions of the disclosure, including but not limited to stiffness, elasticity, degradation rate, adhesion, and swelling, can be finely tuned by modulating the concentration of one or more of the polymers (e.g., HAGM and/or GelMA), the chemical crosslinker, and/or the photoinitiator). In some embodiments, these physical properties can be altered by changing the density (e.g., the number) of functional groups (e.g., methacrylated group) per polymer chain (e.g., per molecule). For example, in some embodiments, different types of functional groups can be used to modify the polymers, such as methacrylate or glycidyl methacrylate, which can be used for photo-crosslinking properties, or polyelectrolytes, which can be used for charge-based interactions and coacervation. In some embodiments, these physical properties can also be altered by varying the molecular weight (e.g., the polymer chain length) of the polymers used to synthesize the hydrogel compositions and/or the nanoparticle compositions disclosed herein. For example, in some embodiments, using a smaller chain length polymer can yield increase stiffness and/or decrease elasticity, thereby yielding a rigid and stiff hydrogel composition and/or a rigid and stiff nanoparticle composition. On the other hand, in some embodiments, using a higher molecular weight polymer (with same density of existing functional groups) to synthesize the hydrogel compositions and/or the nanoparticle compositions disclosed herein can decrease stiffness and/or increase elasticity, thereby yielding a flexible hydrogel composition and/or a flexible nanoparticle composition. In some embodiments, these physical properties can also be altered by the type of drug molecule loaded in the nanoparticle compositions and/or in the hydrogel compositions (external to the nanoparticles embedded or encapsulated within the hydrogel compositions). For example, in some embodiments, if the payload (e.g., a small molecule or a small drug molecule) tends to be crystalline, rather than being amorphous, the payload can form nano-distributed crystalline domains within the nanoparticles and/or the hydrogel, which can impact the release rate (e.g., it can decrease the drug release rate).

Alternatively, or in combination to the polymer concentration modulation, the physical properties of the sealant can also be finely tuned by controlling the light exposure time (e.g., the polymerization time). In some embodiments, the composition is exposed to a light source for about 4 minutes. In some embodiments, the composition is exposed to a light source for about a period ranging from about 15 seconds to 15 minutes. In some embodiments, the composition is exposed to a light source for about a period ranging from about 1 to 10 minutes. In some embodiments, the composition is exposed to a light source for about 30 seconds to 1 minute, 1 to 2 minutes, 2 to 3 minutes, 3 to 4 minutes, 4 to 5 minutes, 5 to 6 minutes, 6 to 7 minutes, 7 to 8 minutes, 8 to 9 minutes, or 9 to 10 minutes. In some embodiments, the composition is exposed to a light source for less than about 20, 15, 10, or 7 minutes. In some embodiments, the composition is exposed to a light source for more than about 10 seconds, 30 seconds, 1, 3, or 5 minutes.

Treatment of ocular penetrating injuries (e.g., lacerations) is particularly challenging due to leakage of intraocular fluid from the injury site. In some embodiments, the composition is a viscous gel that can retain its shape and/or consistency on an ocular injury site without running-off and is able to stop intraocular fluid leaking through the injury site. The viscosity of the composition is an important property that allows the sealant to have a good retention (e.g., no run-off) on the surface of a cornea (e.g., when treating a corneal injury). In some embodiments, once polymerized and solidified, the composition has a viscosity that is greater than the viscosity of the precursor composition prior to photo-crosslinking and solidification. In some embodiments, the precursor composition (e.g., the composition prior to photo-crosslinking and solidification) has a viscosity that is greater than the viscosity of water. In some embodiments, the precursor composition has a viscosity that is similar to the viscosity of toothpaste. In some embodiments, the composition has a viscosity ranging from about 0.5 Pascal-seconds (Pa·s) to about 300 Pa·s. In some embodiments, the composition has a viscosity of about 100 Pa·s at a low shear rate (e.g., at a shear rate of about 0.001 inverse seconds (s⁻¹) to 1 s⁻¹. In some embodiments, the composition has a shear stress ranging from about 1 to 10 Pa at a low shear rate (e.g., at a shear rate of about 0.001 to 0.1 s⁻¹. In some embodiments, the composition includes about 3% HAGM, and the precursor composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the composition includes about 3% HAGM and about 4% GelMA, and the precursor composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the composition includes about 3% HAGM, and the precursor composition has a shear stress ranging from about 0.1 to 10 Pa at a low shear rate (e.g., at a shear rate of about 0.001 to 0.1 s⁻¹). In some embodiments, the composition includes about 3% HAGM and about 4% GelMA, and the precursor composition has a shear stress ranging from about 0.1 to 10 Pa at a low shear rate (e.g., at a shear rate of about 0.001 to 0.1 s⁻¹).

Important mechanical properties of the composition include compression modulus, ultimate stress (or ultimate tensile strength), extensibility, and swelling ratio. In some embodiments, the compression modulus can be varied based on changing the crosslinking density of the polymer(s) used to synthesize the nanoparticles and/or the hydrogel compositions of the disclosure. For example, in some embodiments, higher crosslinking can lead to rigid and stiff nanoparticles and/or hydrogels, thereby increasing the compression modulus. In some embodiments, another factor that can affect the compression modulus is the concentration of the polymer(s) used to synthesize the nanoparticles and/or the hydrogel compositions of the disclosure. For example, in some embodiments, the higher the concentration of polymer, the higher the compression modulus can be. Thus, in some embodiments, increasing the concentration of one or more polymers used to synthesize the nanoparticles and/or the hydrogel compositions of the disclosure can increase the compression modulus of the nanoparticles and/or the hydrogel compositions disclosed herein.

In some embodiments, the composition has a compressive modulus of about 15 kilopascals (kPa) to about 50 kPa (e.g., about 15 kPa to about 25 kPa, about 15 kPa to about 28 kPa, about 15 kPa to about 30 kPa, about 15 kPa to about 35 kPa, about 15 kPa to about 40 kPa, about 15 kPa to about 45 kPa, about 15 kPa to about 50 kPa, about 25 kPa to about 28 kPa, about 25 kPa to about 30 kPa, about 25 kPa to about 35 kPa, about 25 kPa to about 40 kPa, about 25 kPa to about 45 kPa, about 25 kPa to about 50 kPa, about 28 kPa to about 30 kPa, about 28 kPa to about 35 kPa, about 28 kPa to about 40 kPa, about 28 kPa to about 45 kPa, about 28 kPa to about 50 kPa, about 30 kPa to about 35 kPa, about 30 kPa to about 40 kPa, about 30 kPa to about 45 kPa, about 30 kPa to about 50 kPa, about 35 kPa to about 40 kPa, about 35 kPa to about 45 kPa, about 35 kPa to about 50 kPa, about 40 kPa to about 45 kPa, about 40 kPa to about 50 kPa, or about 45 kPa to about 50 kPa). In some embodiments, the composition has a compressive modulus of about 40 kPa. In some embodiments, the composition has a compressive modulus of about 43 kPa.

In some embodiments, the composition has a compressive ultimate stress of about 100 kilopascals (kPa) to about 850 kPa (e.g., about 100 kPa to about 250 kPa, about 100 kPa to about 260 kPa, about 100 kPa to about 275 kPa, about 100 kPa to about 290 kPa, about 100 kPa to about 300 kPa, about 100 kPa to about 400 kPa, about 100 kPa to about 415 kPa, about 100 kPa to about 500 kPa, about 100 kPa to about 600 kPa, about 100 kPa to about 650 kPa, about 100 kPa to about 700 kPa, about 100 kPa to about 800 kPa, about 100 kPa to about 850 kPa, about 250 kPa to about 260 kPa, about 250 kPa to about 275 kPa, about 250 kPa to about 290 kPa, about 250 kPa to about 300 kPa, about 250 kPa to about 400 kPa, about 250 kPa to about 415 kPa, about 250 kPa to about 500 kPa, about 250 kPa to about 600 kPa, about 250 kPa to about 650 kPa, about 250 kPa to about 700 kPa, about 250 kPa to about 800 kPa, about 250 kPa to about 850 kPa, about 275 kPa to about 290 kPa, about 275 kPa to about 300 kPa, about 275 kPa to about 400 kPa, about 275 kPa to about 415 kPa, about 275 kPa to about 500 kPa, about 275 kPa to about 600 kPa, about 275 kPa to about 650 kPa, about 275 kPa to about 700 kPa, about 275 kPa to about 800 kPa, about 275 kPa to about 850 kPa, about 300 kPa to about 400 kPa, about 300 kPa to about 415 kPa, about 300 kPa to about 500 kPa, about 300 kPa to about 600 kPa, about 300 kPa to about 650 kPa, about 300 kPa to about 700 kPa, about 300 kPa to about 800 kPa, about 300 kPa to about 850 kPa, about 400 kPa to about 415 kPa, about 400 kPa to about 500 kPa, about 400 kPa to about 600 kPa, about 400 kPa to about 650 kPa, about 400 kPa to about 700 kPa, about 400 kPa to about 800 kPa, about 400 kPa to about 850 kPa, about 500 kPa to about 600 kPa, about 500 kPa to about 650 kPa, about 500 kPa to about 700 kPa, about 500 kPa to about 800 kPa, about 500 kPa to about 850 kPa, about 600 kPa to about 650 kPa, about 600 kPa to about 700 kPa, about 600 kPa to about 800 kPa, about 600 kPa to about 850 kPa, about 650 kPa to about 700 kPa, about 650 kPa to about 800 kPa, about 650 kPa to about 850 kPa, about 700 kPa to about 800 kPa, or about 700 kPa to about 850 kPa). In some embodiments, the composition has a compressive ultimate stress of about 260 kPa. In some embodiments, the composition has a compressive ultimate stress of about 110 kPa. In some embodiments, the composition has a compressive ultimate stress of about 415 kPa.

In some embodiments, the tensile modulus can be determined by the density of the crosslinked polymers in the hydrogel and/or nanoparticle compositions. For example, in some embodiments, the higher the density of crosslinking, the higher the tensile modulus can be. In some embodiments, increasing the density of crosslinking in one or more polymers used to synthesize the hydrogel and/or nanoparticle compositions can increase the tensile modulus of the hydrogel and/or nanoparticle compositions disclosed herein. In some embodiments, another factor that can affect the tensile modulus is the concentration of the polymer(s) used to synthesize the hydrogel and/or nanoparticle compositions of the disclosure. For example, in some embodiments, the higher the concentration of polymer, the higher the tensile modulus can be. In some embodiments, increasing the concentration of the polymer(s) used to synthesize the hydrogel and/or nanoparticle compositions can increase the tensile modulus of the hydrogel and/or nanoparticle compositions disclosed herein. In some embodiments, the size and molecular weight of the polymer chains of the polymer(s) used to synthesize the hydrogel and/or nanoparticle compositions can also impact the tensile modulus of the hydrogel and/or nanoparticle compositions. For example, in some embodiments, a higher molecular weight of the polymer chains of the polymer(s) used to synthesize the hydrogel and/or nanoparticle compositions can cause increased entanglement, thereby causing an increase in tensile modulus. In some embodiments, increasing the size and molecular weight of the polymer chains of the polymer(s) used to synthesize the hydrogel and/or nanoparticle compositions can increase the tensile modulus of the hydrogel and/or nanoparticle compositions disclosed herein.

In some embodiments, the composition has a tensile modulus of about 15 kilopascals (kPa) to about 50 kPa (e.g., about 15 kPa to about 25 kPa, about 15 kPa to about 28 kPa, about 15 kPa to about 30 kPa, about 15 kPa to about 35 kPa, about 15 kPa to about 40 kPa, about 15 kPa to about 45 kPa, about 15 kPa to about 50 kPa, about 25 kPa to about 28 kPa, about 25 kPa to about 30 kPa, about 25 kPa to about 35 kPa, about 25 kPa to about 40 kPa, about 25 kPa to about 45 kPa, about 25 kPa to about 50 kPa, about 28 kPa to about 30 kPa, about 28 kPa to about 35 kPa, about 28 kPa to about 40 kPa, about 28 kPa to about 45 kPa, about 28 kPa to about 50 kPa, about 30 kPa to about 35 kPa, about 30 kPa to about 40 kPa, about 30 kPa to about 45 kPa, about 30 kPa to about 50 kPa, about 35 kPa to about 40 kPa, about 35 kPa to about 45 kPa, about 35 kPa to about 50 kPa, about 40 kPa to about 45 kPa, about 40 kPa to about 50 kPa, or about 45 kPa to about 50 kPa). In some embodiments, the composition has a compressive modulus of about 40 kPa. In some embodiments, the composition has a compressive modulus of about 45 kPa.

In some embodiments, the composition has a tensile ultimate stress of about 10 kilopascals (kPa) to about 30 kPa (e.g., about 10 kPa to about 15 kPa, about 10 kPa to about 20 kPa, about 10 kPa to about 25 kPa, about 15 kPa to about 20 kPa, about 15 kPa to about 25 kPa, about 15 kPa to about 30 kPa, about 20 kPa to about 25 kPa, about 20 kPa to about 30 kPa, or about 25 kPa to about 30 kPa). In some embodiments, the composition has a compressive ultimate stress of about 20 kPa.

In some embodiments, the composition has an extensibility of about 40%. In some embodiments, the composition has an extensibility ranging from between about 30% to about 60%. In some embodiments, the composition has an extensibility ranging from between about 40% to about 50%.

In some embodiments, the composition is a hydrogel. A hydrogel includes a polymer network filled with an interstitial solvent (e.g., a fluid) which may include water. A hydrogel can change its volume by absorbing a solvent (e.g., when it swells) or expelling a solvent. The swelling ratio of a hydrogel is defined as the fractional increase in the weight of the hydrogel due to water absorption. Typically, the swelling ratio depends on both the polymer/solvent and the elasticity of the polymer. If the polymer is too stiff or the affinity is too low, then the swelling is low or weak. In contrast, low elasticity and high affinity favor high swelling.

In some embodiments, the composition has a swelling ratio ranging from about 5% to about 20% (e.g., about 5% to about 6%, about 5% to about 7%, about 5% to about 8%, about 5% to about 9%, about 5% to about 10%, about 5% to about 15%, about 5% to about 16%, about 15% to about 17%, about 15% to about 18%, about 15% to about 19%, or about 15% to about 20%). In some embodiments, the composition has a short-term swelling ratio (e.g., a swelling ratio measured for a period of about 1 to 6 hours) of about 7%. In some embodiments, the composition has a short-term swelling ratio (e.g., a swelling ratio measured for a period of about 1 to 6 hours) of between about 5% to about 10%. In some embodiments, the composition has a mid-term swelling ratio (e.g., a swelling ratio measured for a period of about 1 to 3 days) of about 7%. In some embodiments, the composition has a mid-term swelling ratio (e.g., a swelling ratio measured for a period of about 1 to 3 days) of between about 5% to about 10%. In some embodiments, the composition has a long-term swelling ratio (e.g., a swelling ratio measured for a period of about 1 to 4 weeks) of about 7%. In some embodiments, the composition has a long-term swelling ratio (e.g., a swelling ratio measured for a period of about 1 to 4 weeks) of between about 5% to about 10%.

In some embodiments, the composition has a water content of about 94% or more. In some embodiments, the composition has a water content ranging from about 94% to about 97%. In some embodiments, the composition has a water content ranging from about 94% to about 95%.

The degradation rate of the composition can be controlled based on the concentration of one or more polymers added (e.g., HAGM and/or GelMA). In some embodiments, the composition has a degradation rate of about 35 days. In some embodiments, the composition has a degradation rate ranging from about 1 day to about 40 days. In some embodiments, the composition has a degradation rate ranging from about 1 to about 5 days, about 5 to about 10 days, about 10 to about 15 days, about 15 to about 20 days, about 20 to about 25 days, about 25 to about 30 days, about 30 to about 35 days, or about 35 to about 40 days. In some embodiments, the composition has a degradation rate of less than about 80, about 60, about 55, about 50, about 45, about 40, about 35, or about 30 days. In some embodiments, the composition has a degradation rate more than about 1, about 5, about 7, about 10, about 14, about 21, about 25, about 30, about 35, or about 40 days.

In some embodiments, the composition has high adhesive properties, especially in wet environments. Typically, to measure the adhesive strength of an optical sealant, an in vitro burst pressure test can be conducted in which a clinically representative incision is made in an ex vivo animal eye (e.g., a porcine eye). An infusion cannula can be placed inside the eye in order to reproduce the physiologic intraocular pressure. Once the incision is created, the optical sealant can be applied over the incision, and the intraocular pressure, required to rupture the sealant, can be measured. Such intraocular pressure can be defined as the “burst strength” or “burst pressure.”

In some embodiments, burst pressure can be determined by density and type of interactions between the hydrogel and collagen membrane, and the cohesiveness of the crosslinked hydrogel patch (e.g., the ability of the hydrogel to store deformation energy in an elastic manner). In some embodiments, burst pressure can be affected by both adhesive and cohesive properties of hydrogel compositions.

In some embodiments, the composition has a burst strength of about 10 kPa to about 50 kPa (e.g., about 10 kPa to about 30 kPa, about 10 kPa to about 35 kPa, about 10 kPa to about 40 kPa, about 10 kPa to about 45 kPa, about 10 kPa to about 50 kPa, about 20 kPa to about 30 kPa, about 20 kPa to about 35 kPa, about 20 kPa to about 40 kPa, about 20 kPa to about 45 kPa, about 20 kPa to about 50 kPa, about 30 kPa to about 35 kPa, about 30 kPa to about 40 kPa, about 30 kPa to about 45 kPa, about 30 kPa to about 50 kPa, about 35 kPa to about 40 kPa, about 35 kPa to about 45 kPa, about 35 kPa to about 50 kPa, about 40 kPa to about 45 kPa, about 40 kPa to about 50 kPa, or about 45 kPa to about 50 kPa). In some embodiments, the composition has a burst strength of about 40 kPa. In some embodiments, the composition has a burst strength of about 35 kPa. In some embodiments, the composition has a burst strength of about 45 kPa.

### Methods of Treatment

The present disclosure presents methods of treating an ocular disease or an ocular injury (e.g., a corneal or scleral injury) in an eye of a subject. In some embodiments, the present disclosure presents compositions for use in the treatment an ocular disease or an ocular injury (e.g., a corneal or scleral injury) in an eye of a subject. The methods can include the steps of contacting the eye of the subject with any of the compositions disclosed herein and photo-crosslinking the composition by exposing the composition to a visible light.

In some embodiments, the compositions can be injected into the eye using a syringe. In some embodiments, the compositions can be applied onto a surface of the eye by using a syringe, a pipette (e.g., a Pasteur pipette), a brush, a dropper bottle or dropper tube configured to dispense a viscous fluid (e.g., having an aperture at a distal end that is large enough to dispense a viscous fluid), or any other suitable device or tool. In some embodiments, the precursor hydrogel compositions can be applied as a drop (e.g., when in a viscous, non-solid state) onto the eye without the need for an applicator. Exposure to visible light can permit crosslinking to provide an adhesive solid hydrogel with biomechanics analogous to the cornea. By adjusting the light exposure time, the polymerization of the adhesive compositions of the disclosure can be finely controlled, allowing for a precise application, as compared to commercially available ocular sealants.

In some embodiments, the methods include applying the composition to an applicator (e.g., a contact lens), contacting the applicator to the eye of the subject, and photo-crosslinking the composition. The first step can include filling the applicator with the composition. Once filled with the composition, the applicator is directly applied on the ocular injury, e.g., by using forceps. This applicator can allow a user (e.g., a clinician) to easily apply the precursor hydrogel composition on ocular injuries with only forceps. The applicator containing the composition can be inverted and placed on the surface of the eye of the subject having or suspected of having the ocular surface injury, without falling off or running off the surface of the applicator when inverted. Due to the high viscosity of the hydrogel precursor composition (e.g., a viscosity similar to the viscosity of toothpaste), leaking from the aqueous humor, for example, can be instantly halted when the applicator containing the composition is placed on the ocular surface. The precursor hydrogel composition can be applied on any size or shape of ocular injuries to stop leaks from aqueous humor. In some embodiments, between about 20 and 200 microliters (μL) of precursor hydrogel composition can be applied depending on the size and the shape of the ocular injury.

When the position of the applicator is satisfactory, the operator can initiate photo-crosslinking to solidify the composition by using a visible light source. In some embodiments, the composition can be photo-crosslinked by exposing the contact lens and the composition to a visible light. In some embodiments, the visible light has a wavelength of about 400 nanometers (nm) to 800 nm. After photo-crosslinking, the composition can become solid and transparent. Once the composition is photo-crosslinked, the applicator can be removed from the ocular surface (e.g., by using forceps).

The applicator can be any suitable contact lens; for example, a hard contact lens, a soft contact lens, or a non-contact lens applicator that will permit controlled application of the sealant on the tissue. Non-limiting examples of contact lens types include rigid gas-permeable lenses and bandage lenses. The applicator can be a contact lens of varied materials, diameters, base curve radiuses, power in diopters and central thickness. The applicator can also have a smooth and regular surface that comes in contact with the ocular surface thereby, limiting patient discomfort and vision loss.

In some embodiments, the composition is used to prevent fluid leakage after cataract surgery. In some embodiments, the composition remains localized over the incision, injury, and/or laceration to seal the wound and form a surface barrier. In some embodiments, upon photo-crosslinking, the precursor composition becomes a solid and transparent hydrogel, forming a biocompatible and adhesive sealant on the ocular surface.

### Ocular Injuries

The present disclosure presents methods and compositions for treating ocular injuries (e.g., ocular surface injuries) in an eye of a subject. In some embodiments, the ocular injury is an injury or trauma resulting from an ocular surgery. In some embodiments, the compositions of the disclosure are used in post-surgical care. For example, the compositions may be administered to a patient after an ocular surgery to deliver a therapeutic agent (e.g., an anti-inflammatory agent or an antibiotic) that may be prescribed to minimize recovery time, prevent and/or treat inflammation caused by the surgical procedure, prevent and/or treat an ocular infection caused by the surgical procedure, or any combination thereof.

Ocular surface injuries can include conjunctival laceration, corneal perforation, scleral perforation, incisions due to ocular surgery (e.g., cataract surgery) or any combination thereof. In some embodiments, the ocular surface injury is a corneal or scleral injury. Conjunctival laceration may occur following blunt or penetrating trauma. Conjunctival laceration is characterized with chemosis and subconjunctival hemorrhage. In such cases, it is important to rule out underlying scleral perforation. The fundus should be examined for any retinal tear or intraocular foreign body. An ultrasound may be done for the posterior segment evaluation.

Corneal lacerations and perforations represent approximately 1 in 10 of ocular traumatic injuries presenting in an emergency medical setting. Corneal lacerations and perforations can include partial thickness lacerations and full thickness lacerations. In addition, adnexal injuries, scleral perforation, or a combination thereof may be involved with corneal laceration and perforations. The standard of care for a corneal perforation include the removal of any contaminants in the wound area, repair of the tear, and maintenance of the watertight integrity of the ocular globe. Corneal perforation may also be associated with or caused by insertion of a foreign body. In some embodiments, the corneal injury is a corneal full-thickness laceration or a corneal full-thickness perforation. In some embodiments, the ocular surface injury is a full-thickness laceration or a full-thickness perforation. In some embodiments, the ocular surface injury is a full-thickness laceration or surgical incision or a full-thickness perforation. For example, the majority of ocular surgeries that require entry into the eye (e.g., cataract surgery) involve a full-thickness incision through the cornea or sclera. Current management protocols for full thickness lacerations including scleral wounds often require the use of sutures.

The compositions of the disclosure can be used to treat ocular incisions or cuts or injuries having a length of less than about 1 mm to about 10 mm. In some embodiments, the compositions of the present disclosure can be used in the closure of full-thickness ocular defects and lacerations and in controlled and long-term drug elution. In some embodiments, indications can include post-operative applications of the biomaterial for drug elution in addition of closure of corneal ulcers, defects and perforations caused by a wide array of insults. The compositions of the disclosure can be applied both under “normal” (e.g., in-the-office or operating room) settings, or under emergency “in-in-field” settings. Various providers, physicians, and, in select cases, physician assistants and paramedics (e.g., in the combat theater) can apply the compositions described herein to seal the eye and elute drug(s) to heal defects. The compositions described herein can circumvent many cases of transplants and patch grafts for corneal melts and defects.

### Ocular Diseases

The present disclosure presents methods and compositions for treating ocular diseases in an eye of a subject. In some embodiments, the disease is an ocular anterior segment disease. Non-limiting examples of ocular anterior segment diseases include conjunctivitis (e.g., allergic, bacterial, and/or viral conjunctivitis), blepharitis, anterior segment dysgenesis, dry eye, meibomian gland dysfunction (MGD), keratoconus, uveitis, pterygium, a cataract, a herpes simplex and/or herpes zoster (shingles) infection, a chemical burn, keratoconus and other ectatic disorders (e.g., keratoglobus, pellucid marginal degeneration), Fuchs' endothelial dystrophy and other corneal dystrophies (e.g., including lattice, granular, macular, and map-dot fingerprint), pseudophakic and/or aphakic bullous keratopathy, ocular cicatricial pemphigoid, or any combination thereof. In some embodiments, the disease is conjunctivitis, blepharitis, a cataract, or any combination thereof.

In some embodiments, the disease is an ocular posterior segment disease. Non-limiting examples of ocular posterior segment diseases include glaucoma, eye stroke (e.g., retinal artery occlusions & retinal vein occlusions), age-related macular degeneration, macular edema, retinal detachment, ocular hypertension, or any combination thereof.

### Methods of Preparing

Provided herein are methods of preparing any of the compositions disclosed herein. The methods include synthesizing the one or more nanoparticles, dissolving GelMA and HAGM in a solution including the visible light-activated photoinitiator (e.g., the precursor hydrogel composition), and mixing the one or more nanoparticles with the dissolved GelMA and HAGM in the solution.

In some embodiments, the nanoparticles are formulated separately from the precursor hydrogel composition. In some embodiments, the nanoparticles are synthesized via a solvent desolvation methods, as described in Example 1. The process of removing/replacing solvating water molecules, by a non-solvent, from the hydration shell of a macromolecule is called desolvation. Desolvation is a thermodynamically driven, self-assembly process for polymeric materials to prepare nanoparticles. Under physiological conditions, proteins tend to bury their nonpolar residues in the core to minimize solvent exposure along with increasing the surface exposure of polar residues. This process not only contributes to protein folding but also increases the solubility of proteins in water. When the critical balance between electrostatic and hydrophobic interactions is disturbed, proteins tend to undergo phase separation and precipitate. A rapid and forced desolvation of proteins thus leads to precipitation (e.g., nanoparticles). Preparation conditions such as pH, ionic strength, amount of desolvating agent (e.g., acetone), concentration of crosslinking agent, and drug content affect the size of the nanoparticles. However, desolvation under controlled experimental conditions can generate homogeneous aggregates (e.g., nanoparticles) with a narrow size distribution which may be subsequently stabilized by cross-linking (e.g., chemical crosslinking and/or photo-crosslinking).

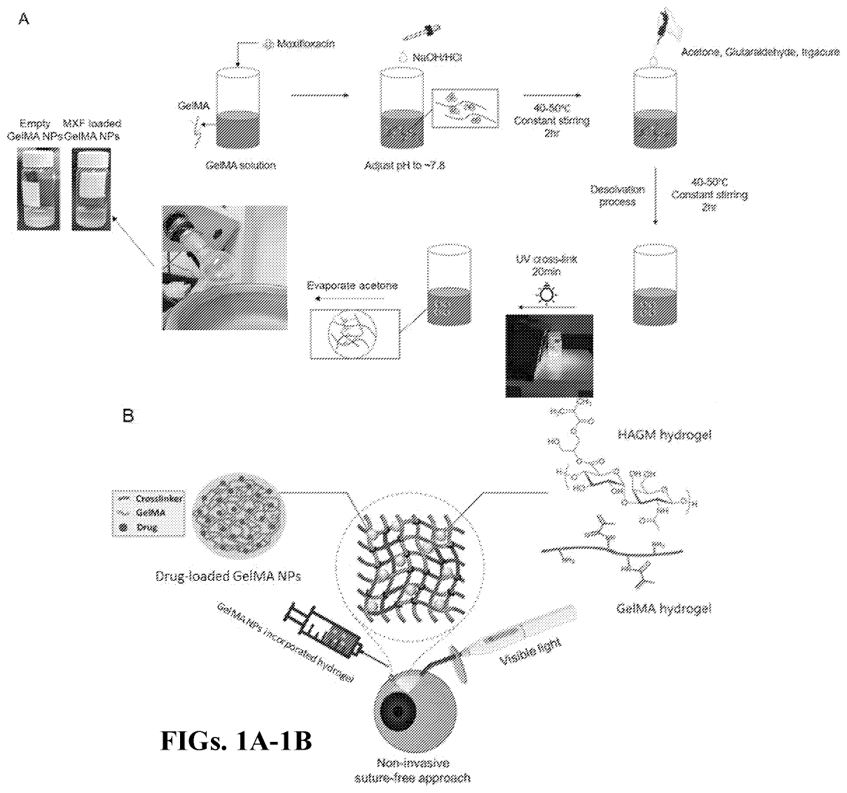

As shown in FIG. 1A, the methods of synthesizing the nanoparticles can include mixing a therapeutic agent (e.g., an antibiotic such as moxifloxacin (MXF)) with the initial polymer solution (e.g., the GelMA solution). Next, the method can include adjusting the pH of the polymer and therapeutic agent solution (e.g., the GelMA and MXF solution). In some embodiments, the pH of the polymer and therapeutic agent solution is adjusted to about 7.8 (e.g., about 7.6 to about 8). In some embodiments, the pH of the polymer and therapeutic agent solution is adjusted with sodium hydroxide and/or hydrochloric acid as needed. Next, the method can include heating the polymer and therapeutic agent solution to an elevated temperature of about 45° C. (e.g., about 40° C. to about 50° C.). In some embodiments, the method further includes stirring the mixture during and/or after heating. In some examples, the mixture is stirred at about 45° C. (e.g., about 40° C. to about 50° C.) for a period of time (e.g., about 2 hours). Next, the method can include adding the chemical crosslinker (e.g., glutaraldehyde) and the photoinitiator (e.g., Irgacure® 2959) to the polymer and therapeutic agent solution.

In some embodiments, the method includes adding a desolvating agent (e.g., acetone) to start the desolvation process. In some embodiments, the solution is further constantly stirred at about 45° C. (e.g., about 40° C. to about 50° C.) for a period of time (e.g., about 2 hours) to generate the nanoparticles. This precursor nanoparticle composition can then be photo-crosslinked by exposure to a light (e.g., UV light) for about 20 minutes. In some embodiments, the nanoparticles are photo-crosslinked by exposure to a light (e.g., UV light) for about 10 minutes to about 25 minutes (e.g., about 10 minutes to about 20 minutes, 10 minutes to about 25 minutes, 15 minutes to about 20 minutes, 15 minutes to about 25 minutes, or 20 minutes to about 25 minutes). Next, the method can include evaporating the desolvating agent (e.g., acetone) from the photo-crosslinked nanoparticle solution. In some embodiments, the desolvating agent is evaporated using a rotary evaporator. Once the desolvating agent is evaporated, the nanoparticle solution can be incorporated with the precursor hydrogel composition.

In some embodiments, the methods include preparing the precursor hydrogel composition by dissolving and/or mixing GelMA and HAGM in and/or with a photoinitiator (PI) solution. In some embodiments, the PI solution is prepared by dissolving one or more photoinitiators (e.g., in powder form) in a buffer (e.g., phosphate buffered saline (PBS)). In some embodiments, the methods include thoroughly mixing GelMA and HAGM in the PI solution and incubating at an elevated temperature (e.g., at about 45° C. to about 55° C.) for a period of time (e.g., about 12 hours or overnight).

In some embodiments, after complete dissolution of the precursor hydrogel composition components, and as shown in FIG. 1B, the methods include mixing the synthesized nanoparticle solution (e.g., the second solution) with the precursor hydrogel composition (e.g., the first solution). In some embodiments, the first solution and the second solution further include a buffer, a solvent, water, or any combination thereof. In some embodiments, the methods include photo-crosslinking the nanoparticle and precursor hydrogel composition solutions (e.g., the second and first solutions, respectively) by exposing the composition to a visible light. In some embodiments, the composition (e.g., the first and second solutions) changes from a solution form to a hydrogel form after photo-crosslinking.

### EXAMPLES

Certain embodiments of the present disclosure are further described in the following examples, which do not limit the scope of any embodiments described in the claims.

### Example 1—Synthesis and Physicochemical Characterization of Gelatin/GelMA NPs

The MXF-loaded GelMA NPs were formed following the stepwise procedure shown in FIG. 1A. To form the GelPatch hydrogel, 3% (w/v) of HAGM and 7% (w/v) of GelMA were dissolved in DPBS solution containing TEA, VC, and Eosin Y as initiators. The formulated prepolymer solution was then mixed with the MXF-loaded GelMA NPs and photopolymerized by exposing to visible light (FIG. 1B).

Gelatin/GelMA NPs formulations were optimized based on polymer type, concentration and crosslinker concentration. Solvent desolvation technique is one of the most applied methodology to form NPs. However, inconsistency in the literatures regarding NPs diameter, PDI and experimental methods exists. Therefore, one focus of this study was to perform a systematic study to check for the impact of variables on formed particle size and PDI.

Ocular nano delivery systems in the form of a suspension can lead to improved drug bioavailability with smaller particle sizes within the range of about 10 to 150 nm. Smaller particle sizes help with enhanced penetration through the tear film and the mucin layer on the eye. Strategies based on the use of NPs for drug delivery can focus on NPs entrapment (chemical and physical) within the matrix of the hydrogel patch and diffusion (i.e., slow release) of hydrophilic drug molecules from the particles to the hydrogel matrix (primary interface) and from the hydrogel matrix to the outside media (secondary interface). In order to have better drug eluting effect, a small NP size is desirable. In addition, a small PDI value ensures that NPs do not aggregate in a solution. Therefore, a particle diameter of about 150 to about 250 nm and a PDI value of less than about 0.2 were considered to be within the acceptable range for this study.

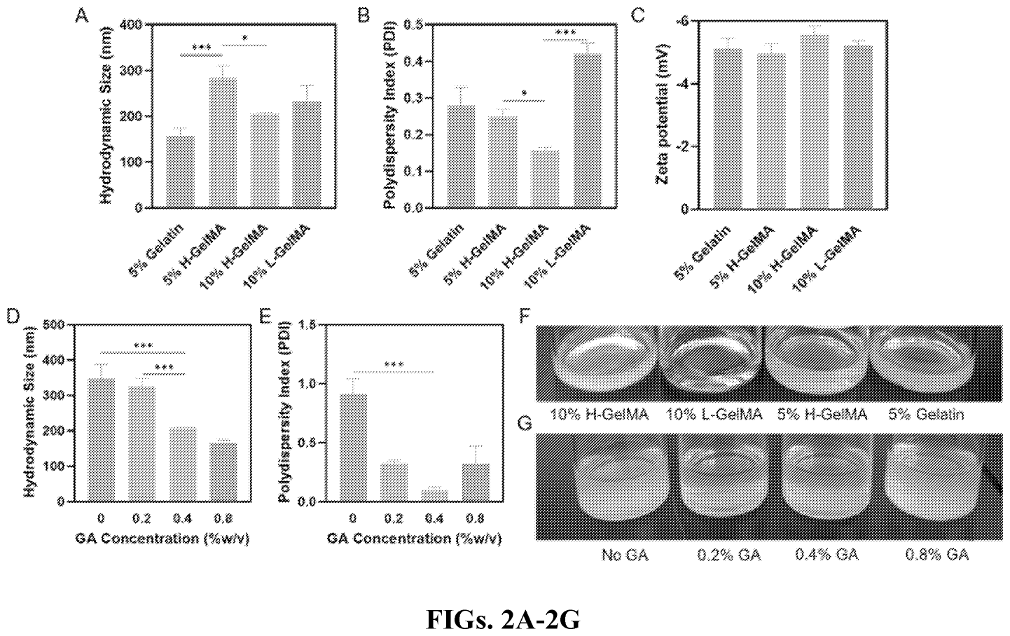

Four batches of gelatin and/or GelMA NPs based on different polymer concentrations and molecular weights were prepared. An average size of 158.5±15.5 nm, 283.8±27.5 nm, 205±3 nm and 232.7±35 nm were obtained for 5% (w/v) gelatin (5% Gelatin), 5% (w/v) high DM GelMA (5% H-GelMA), 10% (w/v) high DM GelMA (10% H-GelMA) and 10% (w/v) low DM GelMA (10% L-GelMA), respectively (FIGS. 2A and 2F). The 5% Gelatin batch had significantly (P<0.001) smaller particle size compared to the 5% H-GelMA batch. However, the PDI for 5% Gelatin was 0.28±0.05, which means that the NP size distribution was within a wide range, which is undesirable for homogeneous NPs distribution within the polymer matrix and drug release. The particles formed by using 10% H-GelMA showed a smaller average particle size as compared to 5% H-GelMA (P<0.05). Also, 10% H-GelMA batch had a PDI of 0.156±0.01, indicating better uniformity of NP size distribution. The particles formed by using 10% L-GelMA were not ideal in both size and PDI (0.42±0.03) (FIGS. 2A and 2B). Based on the obtained results, the formulation of NPs composed of 10% H-GelMA was chosen to be further studied since the size and PDI fit well within an ideal range.

GA is a chemical crosslinker that was used in the NP formulation. In this study, various concentrations of GA ranging from 0.0 to 0.8% (v/v) were tested. The selected GA range was adjusted relative to the applied concentration of GelMA in the solution. The GelMA concentration was 10% H-GelMA based on the results shown in the previous section. The results showed correlation between increase in GA concentration from 0 to 0.8% and decrease in average size of the NPs from 347.8±39.9 nm to 166.5±7.7 nm (FIGS. 2D and 2G). Specifically, an increase in GA concentration from 0.2 to 0.4% resulted a significant (P<0.001) increase in NPs size from 325.8±22.2 nm to 208.9±0.02 nm. Similar correlation was observed with PDI as well (FIG. 2E). An increase in GA concentration from 0 to 0.4% resulted in decrease in PDI from 0.91±0.13 to 0.1±0.02, respectively (FIGS. 2E and 2G). However, at a GA concentration of 0.8%, an increase in PDI of up to 0.32 was observed. In general, the aldehyde groups of GA interact with the hydroxyl and/or amine groups of GelMA molecules and form intermolecular crosslinking within the entangled GelMA chains due to the induced desolvation by acetone. We hypothesize that an increase in GA concentration above 0.4%, apart from increasing the density of crosslinking, also causes intramolecular crosslinking among particles in the solution which appeared as an increase in PDI. Based on predefined ranges for PDI and sizes of the GelMA NPs, GA concentration of 0.4% was selected to crosslink the NPs.

Surface charge density (zeta potential) of GelMA NPs was measured using a Malvern analytical Zetasizer. The results showed that polymer chain length and/or concentrations of polymer had no significant impact on overall surface charge density. Measured zeta potential values of formulated NPs varied between −4.95 mV and −5.56 mV (FIG. 2C).

### Example 2—Physicochemical Characterization of MXF-Loaded GelMA NPs

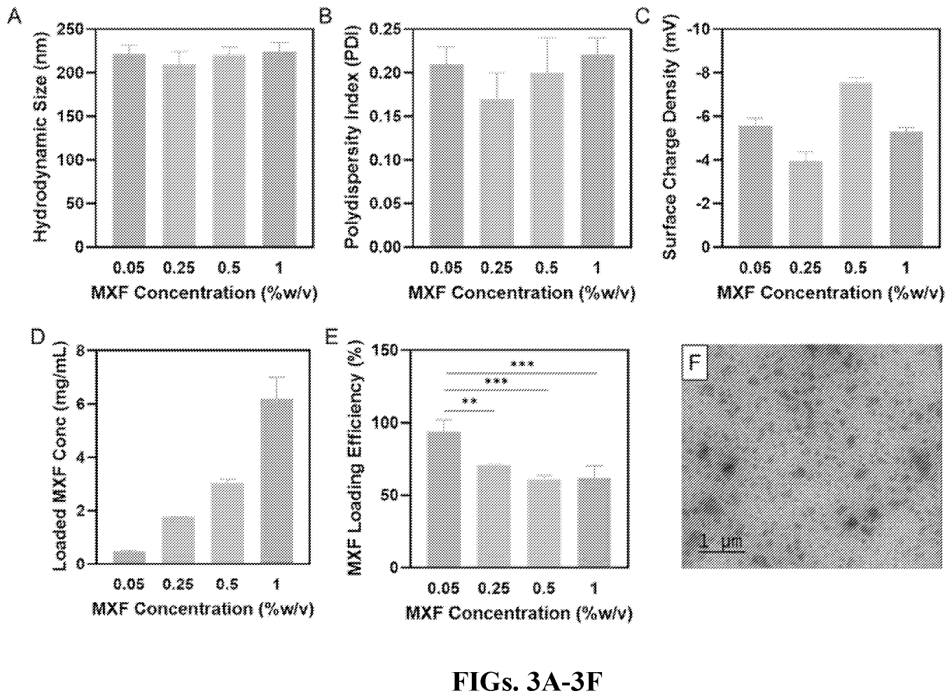

Based on the obtained results from the previous optimization step, NPs formulation composed of 10% of H-GelMA and 0.4%. GA was selected. Loading of MXF into NPs, as an ocular drug delivery system, may increase MXF ocular bioavailability compared to free MXF application. In our study, different formulations of MXF-loaded NPs were prepared based on varying concentration of MXF from 0.05 to 1% (w/v). Results based on physicochemical characterization showed no significant difference on size, PDI, and zeta potential of different formulations (FIGS. 3A-3C). The overall average size of the NPs varied between 210 to 224 nm. The measured values of PDI varied between 0.17 to 0.22. The average zeta potential remained around −5.00 mV for all the formulations, indicating that the addition of drug molecule (at its charged state) did not change the surface charge density of particles.

Encapsulation efficiency of four different formulations based on varying concentration of MXF from 0.05 to 1% (w/v) was analyzed. The results showed that, increasing drug loading decreased the encapsulation efficiency. When the concentration of drug molecules increases, the number of interacted molecules with the charged GelMA backbone tends to decrease gradually and maintains at a fixed level after reaching the maximum loading capacity of NPs. The encapsulation efficiency for the 0.05% (w/v) MXF-loaded NPs formulation was 93.8±8.2% which correlated with 0.47 mg/mL of MXF (FIGS. 3D-3E). With an increase in MXF to above 0.5% (w/v), the encapsulation efficiency dropped to an average of 60.79±2.90% which correlated with 3.04 mg/mL of MXF (FIGS. 3D-3E). Since both batches contain the same number of NPs, it was concluded that most of NPs were loaded with MXF and the maximum loading capacity was achieved at 0.05% (w/v) of MXF. Considering the minimum inhibitory concentration (MIC) for MXF is less than 0.25 μg/mL and 1 μg/mL against Staphylococcus aureus and Pseudomonas aeruginosa, respectively, the formulation loaded with 0.05% (w/v) MXF or above can be considered as an optimized formulation to deliver the therapeutic level of MXF to the injured ocular tissue.

### Example 3—In Vitro Release Studies on MXF-Loaded NPs and NPs Incorporated within Gelpatch

### In Vitro Cumulative Release Study on MXF-Loaded NPs

The in vitro release study was carried out by dialyzing GelMA NPs suspensions against DPBS. The in vitro release profile of 0.05% (w/v) MXF from GelMA NPs is shown in FIG. 4A. FIG. 4A shows that about 32.31±2.56% of MXF was released from the NPs during the first 2 hr. MXF was then slowly released from NPs, reaching to 98.5±0.59% at day 5. With the formulations of NPs loaded with 0.25 to 1% (w/v) of MXF, more prominent burst release was observed as compared with the formulation loaded with 0.05% (w/v) of MXF. During the first 2 hr, a burst release of 44.72±3.12%, 45.46±3.68% and 40.53±1.25% was observed for formulations loaded with 0.25%, 0.5% and 1% (w/v) of MXF, respectively. The burst release was followed by the sustained release of MXF up to 95.52±1.44%, 97.75±1.45% and 84.55±2.16% on day 14 for the formulations loaded with 0.25%, 0.5%, and 1% (w/v) of MXF, respectively. In general, the results showed that GelMA NPs loaded with 0.25 to 1% (w/v) of MXF led to a higher burst release compared to the 0.05% (w/v) MXF. This was primarily due to the existence of non-entrapped/non-interacted MXF in the formulation and only the fraction of MXF which underwent the charge-charge interactions showed the sustained release profile. In addition, the hydrophilic nature of GelMA can facilitate burst release profile via incorporation of water molecules from surrounding aqueous solution and promote the release of hydrophilic MXF molecules which are close to the aqueous/polymer interface of NPs.

### In Vitro Cumulative Release Study on NP Incorporated GelPatch

GelPatch was introduced as a platform for our ocular drug delivery system. GelPatch is composed of GelMA and HAGM. Based on the 1H NMR analysis, the degree of methylation (DM) of GelMA was calculated to be 61% using Eq. 1 and the DM of HAGM was calculated to be 11% using Eq. 2.

The degree of methacrylation (DM) of GelMA was defined as the ratio of methacrylate groups to the free amine groups in gelatin prior to the reaction. The vinyl protons on methacrylamide grafts gave rise to two peaks at 6=5.62 and 5.29 ppm. The peak areas of methylene protons of lysine groups (6=2.75 ppm) in the spectra of gelatin and GelMA were integrated separately. The DM of GelMA was calculated from the following equation.

$$DM ( \% ) = 1 - \frac{{I}_{lysine} ( GelMA )}{{I}_{lysine} ( Gelatin )} \times 100 \% \qquad ( Eq . 1 )$$

The DM of HAGM was defined as the amount of methacryloyl groups per one HA disaccharide repeating unit. The two vinyl protons on methacrylate groups had chemical shifts of 6.16 and 5.16 ppm. The DM was calculated from the ratio of the relative peak integrations of the methyl protons of methacrylate groups (6=1.93 ppm) to the methyl protons of amide groups (6=2 ppm) on HA.

$$DM ( \% ) = \frac{{I}_{{H}_{3}} ( methyl Hs on GM ) / 3}{{I}_{{H}_{4}} ( methyl Hs on HA ) / 3} \times 100 \% \qquad ( Eq . 2 )$$

Based on the results from in vitro release of different formulations, we chose the GelMA NPs formulation composed of 0.05% (w/v) MXF to be loaded inside the GelPatch as drug delivery platform in order to obtain slow release of MXF to the ocular tissue upon hydrogel adhesion to the tissue. As a control group, we applied GelPatch directly loaded with free MXF in order to evaluate the release profile of our GelMA NPs incorporated GelPatch in the release media (e.g., DPBS). The control group showed a 74.7±20.38% burst release of free MXF from the hydrogel patch within the first 2 hr of incubation. The remaining fraction was released within 24 hr (FIG. 4B). However, The GelPatch formulation incorporated with MXF-loaded GelMA NPs showed a burst release of around 29.65±2.65% of MXF during the first 2 hr, and the remaining amount MXF was slowly released into the surrounding media over a time period of 5 days, as shown in FIG. 4B as well.

The release data was fitted based on the non-steady state diffusion model which is characterized by Fick's second law. FIG. 4C shows the cumulative release profile in the form of fraction of drug released (Mt/Mo) as the function of square root of time. The diffusion coefficient D for each GelPatch formulations was calculated based on Fick's second law and the slope of the plotted data (Table 1). The diffusion coefficient for MXF-loaded GelMA NPs incorporated GelPatch is around ⅙ of that for free MXF-loaded GelPatch, confirming that the interactions among drug, NPs and hydrogel network were responsible for the sustained release of MXF from GelMA NPs incorporated GelPatch.

TABLE 1
Diffusion coefficients of MXF from free MXF-loaded GelPatch
and MXF-loaded GelMA NPs incorporated GelPatch.

Formulations

| | GelPatch + | GelPatch + |
|---|---|---|
| | free MXF | MXF-loaded GNP |
| Diffusion Coefficient D | 17.788 ± 1.146 | 3.049 ± 0.446 |
| (10⁻¹⁰ m² · s⁻¹) | | |

Previous studies on the release of MXF and/or highly hydrophilic, charged, small molecules have shown fast release kinetics within the first hr of incubation inside the release media. Our result is the proof of concept that the GelPatch matrix as a secondary interface can improve the release rate of a hydrophilic drug molecule, MXF, from the GelMA NPs, as compared to the freely loaded MXF inside the GelPatch. It is important to note that the conventional ocular antibacterial therapeutics regimen is based on high dose drug delivery within the first 24 hr, followed by lower dose for at least one weeks. Therefore, the developed formulations described herein have the optimal release kinetics for ocular therapeutic purposes.

### Example 4—Mechanical Characterization of MXF-Loaded GelMA NPs Incorporated GelPatch

### Compressive Test

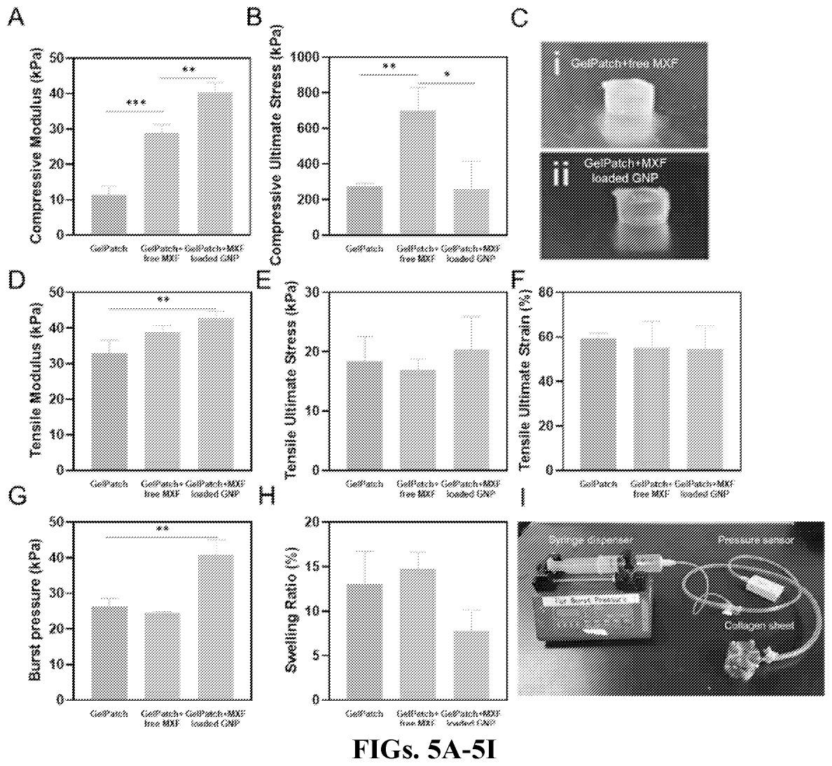

Compressive tests were conducted for three GelPatch formulations, GelPatch, GelPatch loaded with free MXF and MXF loaded GelMA NPs incorporated GelPatch. FIGS. 5A and 5C show the compressive modulus of the three hydrogel samples. The loading of free MXF in GelPatch increased the compressive modulus from 11.26±2.64 kPa (in GelPatch only, without any drug loading) to 28.97±2.38 kPa (in GelPatch loaded with free MXF). The incorporation of MXF loaded GelMA NPs into the GelPatch further increased the compressive modulus significantly, reaching 40.47±2.71 kPa. We hypothesize that the addition of GelMA NPs increased the crosslinking density of the GelPatch formulation, therefore, compressive modulus was enhanced. FIG. 5B is a graph showing the compressive ultimate stress values. Among the three formulations of GelPatch tested, the batch loaded with free MXF showed significantly higher ultimate stress of 701.33±127.4 kPa. This can be explained by the presence of MXF aggregation inside the GelPatch. For the GelPatch and GelPatch incorporating MXF-loaded GelMA NPs conditions, an ultimate stress of 276±15.52 kPa and 261.54±153.70 kPa, respectively, was obtained.

### Tensile Test

Tensile tests were performed for the three formulations of GelPatch described above. FIG. 5D demonstrated that the loading of GelMA NPs increased the tensile modulus of GelPatch from 32.92±3.64 kPa to 42.75±2.03 kPa, which could be attributed to the higher crosslinking density. FIGS. 5E-5F show the tensile ultimate stress and strain of the hydrogel samples before failure. No significant difference was observed in the ultimate tensile stress among the three hydrogel formulations, ranging from 16.85 to 20.36 kPa.

### Burst Pressure Test

Burst pressure tests were conducted to study the hydrogel's adhesive properties to the tissue (FIGS. 5G-5I). The bioadhesive hydrogels of the disclosure may have a superior ability to seal defects or injured sites on the cornea as compared to commercially available sealants, for example. The burst pressure results for GelPatch and free MXF-loaded GelPatch were 26.3±2.3 kPa and 24.5±0.3 kPa, respectively, confirming that free MXF did not affect hydrogel's adhesive properties. After incorporating GelMA NPs into GelPatch, the burst pressure showed a 60% increase to 40.8±4.2 kPa when compared with the GelPatch and free MXF-loaded GelPatch conditions. The engineered MXF-loaded GelMA NPs incorporated within the GelPatch demonstrated a higher burst pressure resistance than commercially available products such as CoSeal™ surgical sealant and EVICEL® fibrin sealant, which are known to have burst pressure values of 1.6±0.2 kPa and 1.5±0.7 kPa, respectively.

### Swelling Ratio Test

Swelling ratios of three GelPatch formulations are reported in FIG. 5H. The GelPatch sample had an average swelling ratio of 13.05±3.69%. After incorporating GelMA NPs, the average swelling ratio decreased to 7.72±2.39%, yet no statistical difference was found. The decrease in swelling ratio can also be explained by increasing crosslinking density in GelPatch containing GelMA NPs. For ophthalmic applications, a small swelling ratio of hydrogel (e.g., between about 5% to about 10%) can prevent building up of pressure and inflammation at corneal injury sites.

### Example 5—In Vitro Biocompatibility of the MXF-Loaded GelMA NPs Incorporated within GelPatch

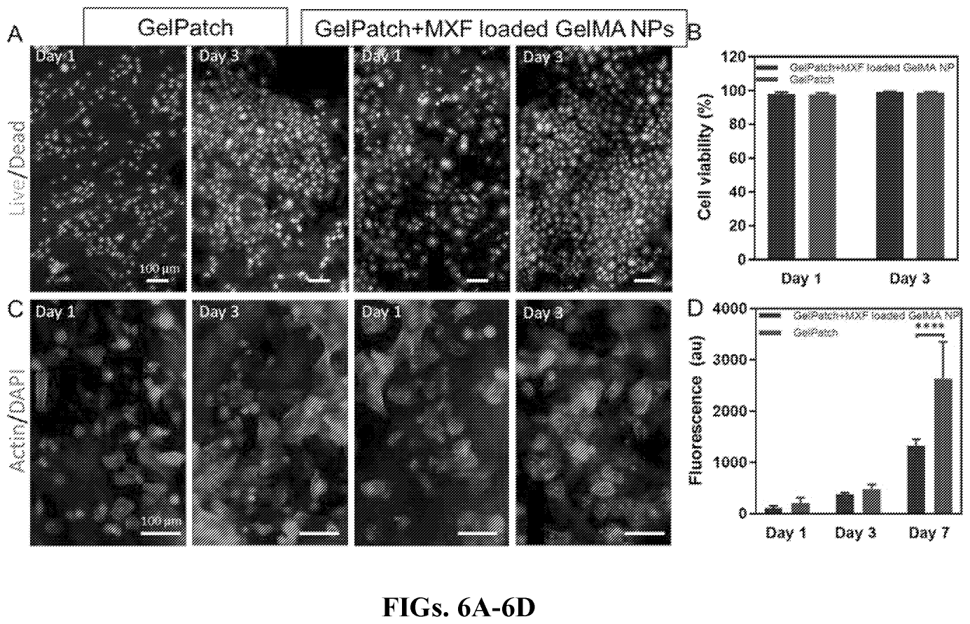

The viability as well as the metabolic activity of seeded cells on hydrogel samples were investigated via a Live/Dead™ assay and a PrestoBlue™ assay at days 1, 3, and 7. These results can be used to evaluate the biocompatibility of GelMA NPs incorporated within the GelPatch. Micrographs of stained cells derived from Live/Dead assay showed a high viability (>90%) of cells seeded on either GelPatch or GelMA NPs incorporated GelPatch at day 1 and 3 which was at the early stage of culture (FIGS. 6A-6B). Fluorescent staining F-actin was employed to help visualize the morphology of cells at day 1 and 3 cultured on GelPatch formulations. FIG. 6C shows that the cells were able to spread, adhere, extend, and proliferate on surfaces of both GelPatch and GelMA NPs incorporated GelPatch which indicated that GelPatch loaded with NPs had good biocompatibility for cell adherence and growth. Furthermore, the metabolic activity of cultured human telomerase-immortalized corneal epithelial (hTCEpi) cells were examined through PrestoBlue™ assay on hydrogel samples. A consistent increase in fluorescence level over 7 days, without a significant difference as compared to the GelPatch only condition, confirmed the cytocompatibility of the GelMA NPs incorporated within the GelPatch (FIG. 6D).

### Example 6—Evaluation of the In Vitro Antibacterial Effect of GelPatch Incorporated with MXF-Loaded NPs

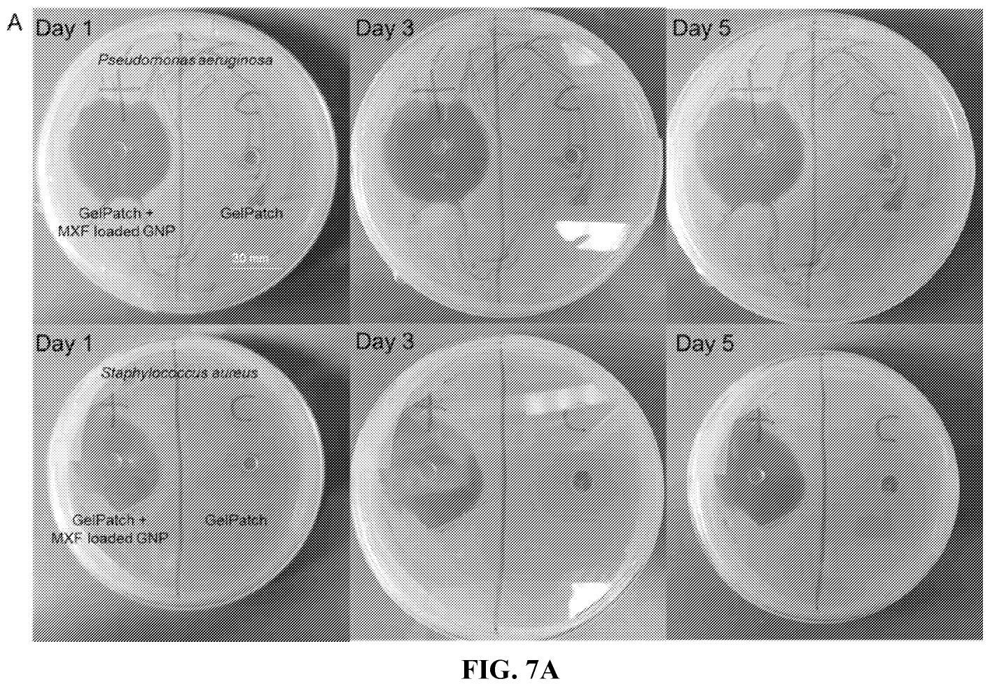

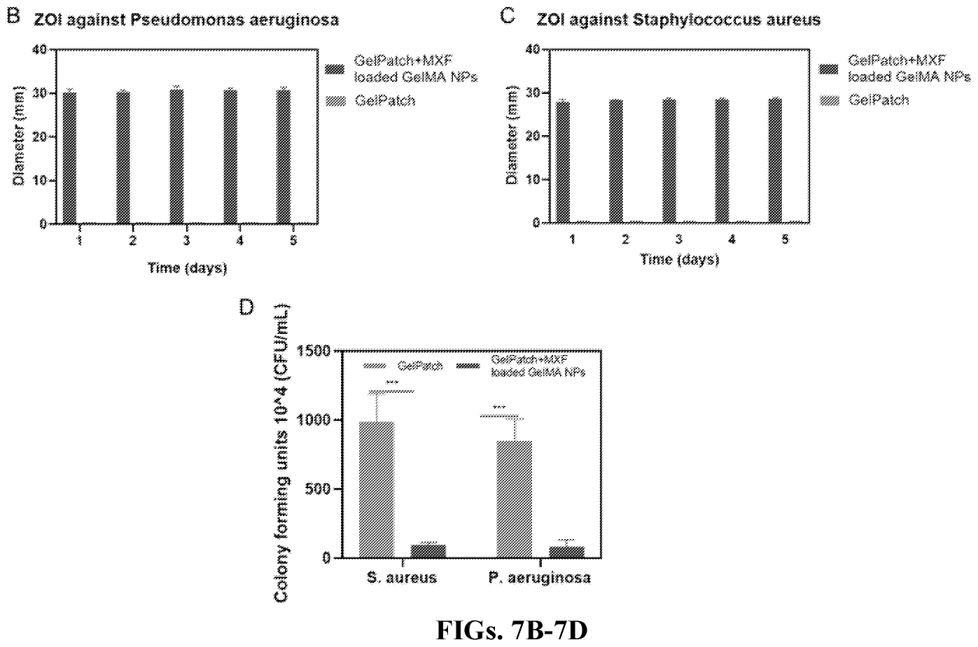

Antibacterial studies were performed using formulations of MXF-loaded GelMA NPs incorporated GelPatch and GelPatch. As shown in FIG. 7A, a zone of inhibition (ZOI) was observed around MXF-loaded GelMA NPs incorporated GelPatch for both types of bacteria Staphylococcus aureus (gram-positive) and Pseudomonas aeruginosa (gram-negative) seeded on agar plates. There was negligible area of ZOI formed around GelPatch samples, indicating that the GelPatch formulation does not contain any antibacterial components. The diameters for ZOI formed were measured by digital caliper every day and reported in FIGS. 7B and 7C. The ZOI shown against Pseudomonas aeruginosa was measured to be more than 30 mm for all measurements over 5 days. This value of ZOI shows the significant antimicrobial effect of the MXF loaded GelMA NPs incorporated GelPatch. For the case of Staphylococcus aureus, the diameter was measured to be more than 28 mm for all measurements over 5 days. These results demonstrated that the antibiotics, MXF, released from NPs incorporated in GelPatch had antibacterial effects against both gram-positive and gram-negative bacteria and the release can last for 5 days. The antimicrobial activity was also verified by counting CFU. CFU for MXF loaded GelMA NPs incorporated GelPatch placed on agar plates seeded with Staphylococcus aureus and Pseudomonas aeruginosa were calculated to be 95±18.38 and 82±50.91 (unit: 1×10⁴ CFU/mL), respectively. About 1/10 of the value obtained for the control group with GelPatch only samples placed on agar plates.

### Other Embodiments

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

What is claimed is:

1. A composition comprising:
one or more nanoparticles comprising gelatin methacryloyl (GelMA) and encapsulating a hydrophilic therapeutic agent;
GelMA;
hyaluronic acid-glycidyl methacrylate (HAGM); and
a visible light-activated photoinitiator.

2. The composition of claim 1, wherein the one or more nanoparticles comprises GelMA at a concentration of about 5% (w/v) to about 30% (w/v).

3. The composition of claims 1 or 2, wherein the GelMA present in the composition comprises a degree of methylation of about 40% to about 85%.

4. The composition of any one of claims 1-3, wherein the one or more nanoparticles is chemically crosslinked and photo-crosslinked.

5. The composition of claim 4, wherein the one or more nanoparticles is chemically crosslinked with glutaraldehyde at concentration of about 0.04% (w/v).

6. The composition of any one of claims 1-5, wherein the hydrophilic therapeutic agent is a first therapeutic agent, and the composition further comprises a second therapeutic agent.

7. The composition of any one of claims 1-6, wherein the hydrophilic therapeutic agent is present at a concentration of about 0.01% (w/v) to about 1% (w/v).

8. The composition of any one of claims 1-7, wherein the hydrophilic therapeutic agent is present at a concentration of about 0.25 μg/mL to about 5 mg/ml.

9. The composition of any one of claims 1-8, wherein the one or more nanoparticles are encapsulating the hydrophilic therapeutic agent at an encapsulation efficiency of at least about 85%.

10. The composition of any one of claims 1-9, wherein the hydrophilic therapeutic agent is an antibacterial agent.

11. The composition of claim 10, wherein the antibacterial agent comprises antibiotic drugs, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, sulfamethoxazole, chitosan, or any combination thereof.

12. The composition of any one of claims 1-11, wherein the one or more nanoparticles have a diameter ranging from about 150 nanometers (nm) to about 250 nm.

13. The composition of any one of claims 1-12, wherein the one or more nanoparticles have a polydispersity index (PDI) of less than about 0.2.

14. The composition of any one of claims 1-13, wherein the composition has a compressive modulus of about of about 15 kPa to about 50 kPa.

15. The composition of any one of claims 1-14, wherein the composition has a tensile modulus of about 15 kPa to about 50 kPa.

16. The composition of any one of claims 1-15, wherein the composition has a burst pressure of about 10 kPa to about 50 kPa.

17. The composition of any one of claims 1-16, wherein the GelMA is present in the composition at a concentration of about 3% (w/v) to about 14% (w/v).

18. The composition of any one of claims 1-17, wherein the HAGM is present in the composition at a concentration of about 0.5% (w/v) to about 3% (w/v).

19. The composition of claims 17 or 18, wherein the composition has a sustained drug release profile over a period of at least about 5 days.

20. The composition of any one of claims 1-19, wherein the photoinitiator comprises Eosin Y, triethanolamine (TEA), N-vinylcaprolactam (VC), or any combination thereof.

21. The composition of any one of claims 1-20, wherein the composition is in a form of a solution or a hydrogel.

22. The composition of any one of claims 1-21, wherein the composition further comprises a pharmaceutically acceptable carrier or excipient.

23. The composition of any one of claims 1-22, wherein the composition is formulated for topical use.

24. A method of treating an ocular disease or an ocular injury in an eye of a subject, the method comprising:
contacting the eye of the subject with the composition of any one of claims 1-23; and
photocrosslinking the composition by exposing the composition to a visible light.

25. A method of preparing the composition of any one of claims 1-23, the method comprising:
synthesizing the one or more nanoparticles;
dissolving the GelMA and the HAGM in a solution comprising the visible light-activated photoinitiator; and
mixing the one or more nanoparticles with the dissolved GelMA and HAGM in the solution.

26. The method of claim 25, wherein the solution is a first solution, and wherein synthesizing the one or more nanoparticles comprises: mixing a chemical crosslinker and a photoinitiator with a second solution comprising GelMA while constantly stirring; and exposing the one or more nanoparticles to an ultraviolet light, thereby photocrosslinking the one or more nanoparticles.

27. The method of claim 26, wherein the chemical crosslinker is present at a concentration of about 0.1% (w/v) to about 1% (w/v).

28. The method of claims 26 or 27, wherein the chemical crosslinker is glutaraldehyde.

29. The method of any one of claims 28-31, further comprising mixing a therapeutic agent with the second solution prior to mixing the second solution with the chemical crosslinker and the photoinitiator.

30. The method of any one of claims 25-29, wherein the first and second solutions further comprise a buffer, a solvent, water, or any combination thereof.

31. The method of claim 25, further comprising photocrosslinking the solution after mixing the one or more nanoparticles with the dissolved GelMA and HAGM by exposing the composition to a visible light.

32. The method of claim 31, wherein the composition changes from a solution form to a hydrogel form after photocrosslinking.
